SEQUENCE LISTING (22 sequences)

SEQ ID NO: 1 - DNA, 1000 bp, Arabidopsis thaliana
  misc_feature (1)..(1000): Ceres PROMOTER PT0695 As Stated in Report Number 211
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 2 - DNA, 435 bp, Arabidopsis thaliana
  misc_feature (1)..(435): Ceres PROMOTER PT0879 As Stated in Report Number 225
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 3 - DNA, 1000 bp, Arabidopsis thaliana
  misc_feature (1)..(1000): Ceres PROMOTER PT0738 As Stated in Report Number 246
ctcataatctgaatttttcaattgcatgtattagctagagaacaaaaaatcggtactgatttgaaggcctaaaaagtctatgttgtttagaacgttcaaatatcttaccatgggaatgaaagtggacacgagtcatgtgatcatccttttggcaccattgttttgtcttctaatcgaacacgtagtgtcattttcatatcttcaatcgtatattttgttactactagtcaagtttcttcttatataagaaaatgggacatgatggattatgatttaagtgttccaacggtaggtaatgaagttaattcgactttttttaatcattttaataatgagatgtttggtgcgtcatacataaaacacaacatcaggtctcatcattaggcccttttttagtaatattcaatgttttcttttacattttcatatcacggatccaagtaattaagaaaacaaaaaacaaaaaacgaacctaatttatatacacatttcaacatattaatagttggtagtagtaggagataaattgatatctagttaaataatcttataagttttgagataattggtcggcgttacgactgtattatatacttctttttacccggttagtattttcctaatgagaaaaaataaataaatcgattcatgaaataaaataattttgaaggatgatgacatgatgtaatagagttaagagtagataccatgaaacaagggtattttattttcaaactttctaaaccataaagcacaacacaacagtattggtgtcctaactttatctgtcttctaataaatattaaatagaagaaataaaatcgaaacatgttgaccataaaaaaggttttgtttgttaaacaacccggtttcaaatctaacacatgaggttgcttctcatacgttcttaactagtagcagcagagttatctatataaactcttctttgtcatagaacaataaaaaccaaaaatcaattttcagaaaccatttcgtgaca

SEQ ID NO: 4 - DNA, 960 bp, Arabidopsis thaliana
  misc_feature (1)..(960): Ceres PROMOTER PT0834 As Stated in Report Number 247
attggttcaacaaccctcgggggattagggtttaggtaagtataagaaccctaactttgtaagtatagcaagtactgaatgatgtttgattgttaggtttgtaatttttgtgtcacaatgttaaaaggtatttcacagtggtaaggtgctttatttgcgatttgatttggcataggaccttataagttctgacactgtagagaacctatggaaccttttcttatgtgatactataagaaactcgtgattttagctgcatctgattgacgttttatgttttgataatagtttttggagacatgttgtcaaagtagaagtaaagacaaatgtttctttaattcgtcggactagaaatcaaatcttcaaaagccatttttgtcgtcccaatcgactttcaaaaaagctttaaattagaacgacacaacaatgaatgatttaacccacaaaaagcttgaatccaaaaggtttgacttttcagaatttagtcttaaaattccgtaatcctatatattaacggataagatccaacagagcttcagaatttgtccttaactcatattcttcttattttctatattataattcgagggttacgtgtcatatgcctactactgacatcacttgcgtcttccttttctcttgtcctctttcgtaatcattaaagtaagtaccttttaaaaaaaataaagaaaccaacggtgggagaggaagcagagagagagaaaataaaaaaaacagaaaacgagaaaaccagagattcatcaattcattgattctttagttcttcttcgtccgtctaaccttagaaacttatagctacagcgagagaagacgaagaagaataagaataagaagaaagaaaattgaggcctgcagggccagtgcactgggatccaacaatgtcctccgactcgtccaagatcaagaggaagcgaaccgctccgcacaaaaagtcacaca

SEQ ID NO: 5 - DNA, 1001 bp, Arabidopsis thaliana
  misc_feature (1)..(1001): Ceres PROMOTER PT0746 As Stated in Report Number 254
aaagatcttgatggaaattaccgaggcatatattttatctatatataacaaattaaaatatcaaatatatatacaaaagctcttggtcgtttttttttcttcttttttggtacaacattcagaagctacaaactcaaatacggagttggcgtatacatggaccaaggatccaatagaaacatgtatagtattaattgagaaatacaatgtgaatagcaataattatttttaaataaattactgaaattgacagtgaaaaaaaaaagaaactactgaaacagcaaattgaataccctcatcaaagggtgaaattgttagaaacataattgaagaatcgtggaagagatcacatggttatgatttgatctttaatacaacttggtgttaatgattttgattttcaatatagaaaagattattgtcgcttattgaaccacaattaacaaattacgttactaattgtattttacatatactctaaaatcatcaaatagaagatgtagttagttaaaattgtcaaatagaagatgtagttagttactaaattctaatctggacaaaaataaagatgtggttcatacgtgtgagaagagacactaccattcttttattacatttcgttgtagtgaggctttttctgttcagtcttaaaatacaaattaaactgtccagtgtccactacactaatcacttcatgctacggcccgctaattaatgtttaaatatgtattgatcggatcagaaagaatatttgtatgatcaaaaaaaaatctgttctcttcctcacagcttttctctcagatctgtttgttcgggtttatttttgctctcttgcttagttctgtttttcactcaactacaattctttcttaagtttctatggttagatgaacttagactgttattgtttttggaaaaatccgtagatctatccgtctcaatactttgataaactaaaatctgtttctttttcttataaaattgcagattttgtatcacac

SEQ ID NO: 6 - DNA, 1000 bp, Arabidopsis thaliana
  misc_feature (1)..(1000): Ceres PROMOTER PT0607 As Stated in Report Number 299
agttaatgatgaaaattcaataattaagttaatttgttttttgattaaaagaaacaaaaagcatcgtttaagaaaataacaagaaaacaaatcatacgtgattttacaaatttatgctcttgtcccgtctcattaccaagcgggaaattaaatcttggtctagatggaaatgcgtttcataataaaacaagaatttgaaatcggtaatgaaacaagatgggacgaaaactgagataggatatggaatggaaatctggtccacacatttatcggaaggtggactttagctttcaggaattaaagangttggatagattttccgtgagtacaccaaatttgtagtattaacagaagttgaaattacggtgtgaactctagtagctacaactcccaagtctttaactacatttgatatataatgttttggaacattcgttctctactactgatcaccacgtttacatcacttttctttacttggtctaggtaatttaatttagtactcaaaggtatatatatgttgtactgtcttcgtctatagagactaccacgtccattcttattttgttggacggtccatttttaaggatcaagaagataactaaaattttgactttttaaatgaaagcggagaaaaccaacacatttttatgcttaattaggtcttttgctaaacttcacaagagaatcgagttatgagactacgtacgatgatgtgataaaatctctcagaataaagttttcatgacaatgacgtatctcctaacttataatattagtaaacaaaatccatttgattatcagaagattagaggacacagctaatgctatgctaatgtgaactgcgcgtaagacgcgcttaatcatttgtcaatgtgtaaagcgaaaaatattaaaaatatgaaacacgagtccgagtaaagtgacgatagtaacgacgtaagacgagaaactctggtctacacctttataaagagataagaaatctctctctccctca

SEQ ID NO: 7 - DNA, 579 bp, Arabidopsis thaliana
  misc_feature (1)..(579): Ceres PROMOTER PT0861 As Stated in Report Number 300
agaaaaaaaaactcaattcttacaaaggaaaacatctggtgaaaacaatggccaactaagttgtaagaccatagatagtaaactatgtgggccatcaattaatattattcggacagttagtgaaaattagcctaattaggtattctaataacatttttcccattcatcatttagcatatgtatgtagggtctcgccatatattagtatacatttttatcccctgggaggatgtgtgtgagctgtgaaagactgaaaagagttggtaaagtagtgggtttagtttctttttccagtaaaagtggttagctttttccctctcgtgctggacctatctctttggttttatcatcatacacttcgttaaagaatagcgtaaatagtaaactcgagagtctaaacaaattattggcaaaatgtgtaattaaatatgatggaacggccgacggcggctaaaacgtgtatcagtgcatgcgtagtaaagccatccctcattcatttgtttaaatatcaatatagagcccaaaaacaataaaggaagcattaaacacaagaatagagagagaagaaagaagaagacg

SEQ ID NO: 8 - DNA, 1000 bp, Arabidopsis thaliana
  misc_feature (1)..(1000): Ceres PROMOTER PT0760 As Stated in Report Number 318
tacatacataacatgcgaatgtcttgtgtctgtgtatatgatatctaatgtatgggaaaacttggaatataggttttattttcttctagacaagatcatgaagtatagtacgtatcttttatgatcatctttttggtcagacgaaagaatattagttttatataatacataatttgtgatttgtttttctatcctctataagcacaaacatatactatcagattatgacacatcaacagtgctaatataacattaggctgtaaaatcaacttaaatattttgtaacacttttctcttaactattacaataaacgtatagctgttagtttgcaaggacagcgttaacaacttattgtatcatagtattgattcaaaacgttaattactgtgataactataacttattaactgaaatgcagacgttgacagacacacaagaattgaaatgtagaaacgcgtttatataagagaacgctaaatatataaatttcggacttttctagtgaaactctattagtcttcttttttttctttggtcaaaaaacttccaatacgaaattcgaacaagaagtataattagtgatcaataatgcgaatgatgcaataaaaagtcaaatgaaatgaaattgatgaatttaactgttatataaaattataagttgacagaatgaaatagtcatataattctagttctagacgtggatagcaaaaactttgtcgcccatatgtatataaaatttgtactctagttatcttttaagtatataaggcttatattatacatcgaagtcaatggttctttgtggtctgggtctataatcacaggtaagcacatcgtcatcgatttcattttcaaagtcaagaccaatctcgctctctatataattgttaaaaaccatctttctaattaaaatagaaagagtaaaagccaagtaacaccattttaaaaggcttcgctttcaattcagaaagaagagaatagagagcttcttcttcttca

SEQ ID NO: 9 - DNA, 436 bp, Arabidopsis thaliana
  misc_feature (1)..(436): Ceres PROMOTER PT0878 As Stated in Report Number 319
ataacaagtagcggggatagctcagttgggagagcgtcagactgaagatctgaaggtcgcgtgttcgatccacgctcaccgcatttgtttttgttttttcgcactcgattagtgggcctggacttcgaacaaaatattcgataaggtccatttattttcggatttggcccatattaatcaaataagttgtacggagtattttaatagctcaacaagttctgacaatccttatagaacatggccgatcggatctataggttgattgattgcttttactttgagattatttagagatttatataatgcgacttgcaatagtacttattgacttacattcaaaaaatttgactttctaagccaaagacattactatatgtaaaagcttcagatcgtgaatcttgattaaaattatctgagtagcaagaattcaaaagaa

SEQ ID NO: 10 - DNA, 999 bp, Arabidopsis thaliana
  misc_feature (1)..(999): Ceres PROMOTER PT0513 As Stated in Report Number 137
ccaaaatatctagagacccgtggttctatatgtacattttaatttttaatttgctaaagtcagccatgaatactatagtatacctagtcagatttgatgatgatggttgttaaacccaccgctcctcctagtcaaacatcttctacaatttccaaacacattcaaaaaacgcagaaaaaaaaaaaaaaaatgtgtatatgtgttacaattcttaaaagaaaatgtaaagccagataattgttacaagagatgtacaaagctccaagtgtgaagaacatcaacatgaatatgatccatgaaatctaccaaatttccgtgcttgcactttcccatacatttttgtttttcacctaactcttacgaacctaaaataaaataaaatacaaaaaaagggagaaagatttgagcacttgtcgacacatcgtagcatcctacctctgccgacacgtggctcgagacaaatacggaactcccggtcggaacagaaacaaatacttattttgactacctgaaattcgacacgtcatcaccttctagactcaaaagagtacaaaggccaatgagactctaccattcctaacgaagtgtcacgtcacttcctatcaaacctttccttaaatacgttcactgccgcgtcactcctcttttttggtcacgtttcctatttttgtactaatacttcttcttctctctacacatattttgttggtaatcacatcgaagattcgaaattggatcacatttggtaatcaacttttttttccaaagtctccacgtgtatggacgattacatacgtacacttatttattttcgctttggtggataaactcgatgttaccaaaagtatacactatctaattaccgaaccaaaattaatctgaaacccttctatatatatatactttctttgcaataacagagtgaaagcaaacattcaaaaaaaaaaaaaaaccattttttttttcttactcaaatcttttttagtatcattctccaaa

SEQ ID NO: 11 - DNA, 1000 bp, Arabidopsis thaliana
  misc_feature (1)..(1000): Ceres PROMOTER PT0590 As Stated in Report Number 152
attattcaatttaataaaaattgagtcggccaatttaatgcgagacttctgtacaacgaccctaaaagtgggtttgataaatgaaacatattgcaacaaaaaaatactagtaataatgataaaatagtaacatgtcgtggcgcattgaatatcctacgaaggtttagtgtttacttttaaaaaatcctaatatgatactagtacatatagctagcttgccttgcttatgctattgcatagtctgtattaataaatgatgttatacatttcgatagagtaacattttgggaacatgagtgaacgtgcttgaatcttcgtgcccttgacgtcagaagctagtaattttaaatactaattaacattcatacaaattaacagatacaatgtactatatcataattcgtttccgtaacacaacgcaacaatttgaaagtagatgtactttagtacttagttagtgtgcaccaaaaaaaaaagatgtagttagttagtaaggggttaaatgttttaatttattaagaaaacttaaattcattaaatgttagaaaaagtctaattagtttatattcgaacactgtgctcaaaattaaaaagtcaactattttagactatagagtttattaattaataataaattcgataaatcaccgtattattttcttcaacgacaagtagccgtgaagacacgggagcgaagagagataaacagaagatgaagaagaagatcaatgtcataatcttcagggagataaatccgtaatctttattaatcaaggttaatccttttttttttcttcatcttaattctttgcgtcttccttttctatttatcacgagatctgtctttctttttcctcttctttctctctcttctctctgaagacagtacttgtttctgtccggcgttaaaagcttcggtggtggtctcttgacttctctgagaagaagaaaaggaagctgagtctcattttagattcagctcacgaggaagtgacgacga

SEQ ID NO: 12 - DNA, 633 bp, Arabidopsis thaliana
  misc_feature (1)..(633): Ceres PROMOTER PT0850 As Stated in Report Number 191
aaacctgttattatcggtggcgtacccaatcaattaccgactgcagccggtcgcagcgtctattcagccgacctaagaaccggtatcgccggtatcaataattgctgatcttttccttctcttcttccccagattctcaagtctctctaacttggagaggagatagaagacatctcatcatcaatctccggatccaatccaattctgcatcgactcttcagaggtatttccgatgaaccattattttccccttgtagaaattattctcctttctcaattgatgattgtccacatagctagattttgtgtaaagagatattttgtattatagattgcatctactgctattgtttcccgattgattgaagcaagtagacaagaaaaggattcggttttgtctggttctgaaatttccattggttccaaattgtcatccttgatgaagaagatactttgctatctattagaaaccagaatggctcgtgtattgcctgattctttgtttaccttttgttgattagagtgattttgcaattggagatgttagatgttctctgtttgttttgtggctactaacccaagaaggtttcttttcagttttattgggtcattggagatttttttcggttgagg

SEQ ID NO: 13 - DNA, 999 bp, Arabidopsis thaliana
  misc_feature (1)..(999): Ceres PROMOTER PT0723 As Stated in Report Number 212
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgtattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaa

SEQ ID NO: 14 - DNA, 1000 bp, Arabidopsis thaliana
  misc_feature (1)..(1000): Ceres PROMOTER PT0769 As Stated in Report Number 258
ggaagctcaccatgtcttcttcgatttctttccgtccaaattgaatggactgtcgctttaaatatacgtagcgtaaaatgaattgatgcgtcttcgagaactgagaagtggagacaatacttagcaaagcagagtaatccaaagtaaaatcttgcatgagaattcgcttggcaagatgttcccagatttgagccgagaatgtgcagctgaaaaacaaatgatcccttgtctcaaatggaacttgacaaaacacacatgttggattgagattcgatgaccaagagcacatttgatcacatgtcacgttgaagttggttttatgtttctaaaaataatgccaaacaataaatgctcgaaactgaagtccacctacaagttttacgagtaggtttgtcatatcataaaaaacaaaatctacgctgtcgatgagatgcaatcatgcaaggtcgttttaaaataaccgtttgtttgtggaagacgtactttaggtagtctttgaataattatttcacaactagtttatattttcacaaatttaatatttaatcttgcaaccagcagcctcatatataacaatataaatatacattttagaaaaatggccaaaacttgtatatatgaaaaaagccaagtccatcgctaaacttactgatcagcatagcagtctgttcatcttcagaggtcagctcttttctctttccctcataagcatgcttacttcttattcgcaccaatgttttaaccttaaataagatcttatcgaaatctattatttatgtgaacgtaatgagcatagttttagatatcctcttacagtagttccgtttctgcaattggtgatcttaaatattgtgttggttcttcgcttgacaatatgttttgtttttttaatattcccaatttaattaaggaacacatgtggatattgatgcagattattgtactaggaaacacaagaaccacttcatatccagctgaatattgataacatcagttcaag

SEQ ID NO: 15 - DNA, 1000 bp, Arabidopsis thaliana
  misc_feature (1)..(1000): Ceres PROMOTER PT0614 As Stated in Report Number 293
ttacaaagtcgcgatgcaacaacctttgtactatatgatgagaatataatataaaaatgggcaacaagaacaaaagaaaatagagaaaaaaaggaaaggataatcttccacctaaacaagtccaaagaagattgcaagttgcaacatccacttgcttcgtccggcgtaagctctttgataaggtctctcaccgtcctccgactttctctctccatacatacaccacatttacatacgccatttacacatatatgacaaacccatcaacatctgtgcatgcacgtgtgctcatgcatgtatgtagtttttacttcgccatgatgaccatgtctatcttaaaatttaccacaatcaatcaatcatgcgcgttacagtcgtgtgatattcaaaaattgtacatgttatttctcataactatcactttagtagattaagatcagaatgtgcatataataagtaaattttaacaataacagcaacatattttactataactaatgatttatttaaaaaaaaaagataaaactaattctgaaatttcagttttttctatgaacaatttattaacagtattcatatgtagataaaccttgtagattctataatataaaatttatcaagatggaaatgagggatctatagaatacaattaattttggtgatatatattcaagataatgaaggaggcgtctgaggaaagaaatcatgggggcataagatggtacaatgtatcatacatggtcacacacatctatatgatacaaatgcgtctatatacacaactgtttctatatacatacaaacacaaagccttcacatccccagctatctctatccatctatcttcaaatatatatttttaaaaacacacaatgttcatatcttattgttattgttataaaataaaagatgatcatactttttaaatttctcaaacaaaaccaacttgacaaccgacagcgaacaagatcaaaaagctagctctcttcttttctcatca

SEQ ID NO: 16 - DNA, 1000 bp, Arabidopsis thaliana
  misc_feature (1)..(1000): Ceres PROMOTER PT0621 As Stated in Report Number 294
tttgatctaaaaacataagaaaataacattattagtaagtaaagacacttggtttaagtagtaaactccctaatggatctagtttttcactttccatgtcattttcaaatattatatcgactaaaataggtgataacaaaaaagtaacttatagagagaaaaaaaaattagttcatgttggagacataacttttgctaatttaaatcaaagatcaaatgaatttggtttgcttttgggactatagtgaccacacatataataaatagtgtgattccacgagatttgttattggttaaaaggtaaaattgaaaaattgcagatcattttgtctttgtgcagattgccaatttttattcccaacggattggatttgccaattcaaagtcaacagaaattgtaattaaaaaaaaaaagaagcatatttgagttagtgatcaggatagacctatgatggctaaagactttctagaagaatatgaatatcaaagtagtcgtctataaatgatgacggctaagactagtaaacataaatttgttaattttttcaagtaattttttcttcgtgtgatcacatatcattaacatgttccaaattcatggtcctcttatcttgaataagattttagtatgtattgtaaactagctcaaaacattaacatttctgtaataaacgagtgaatgaaacttgccggtgaaatagtaaattataaaaaagagatatgaaaatgaccattttagatgaaataaaagatacttttcatacatacatatacagagtttaggaccatttcaacaacttcttcgaagtcaccgaactattttgatttgtatgacctttcacttccatctccaattggaaacttctgaccgtccacgtggcattcaataagtaacacatttgcttatgacatatcccctaacttcctcgcttcttctccacgactttttcaaacttttcaaaacctccctataaacttcagatccttattcctcacaata

SEQ ID NO: 17 - DNA, 1000 bp, Arabidopsis thaliana
  misc_feature (1)..(1000): Ceres PROMOTER PT0693 As Stated in Report Number 295
tctcggatatcccaatctgagtatggttttaggtcaattcaacatagtattattttgtcaacaagcaccattatagcaaaaacaaagatgtatcaaagaaaagtagcgttaggggttggtcagaaaaagacatgtttgcagtgtaagaaatgacgtggacacgtggaatcatacgcaatgcaacatgtcatcattaaggtccattgttcaacttacccattgagaacaaaactagaaaaattatagaccgttggatcatctttaaggaccatgtctgattcacgtatctctctcccacttggaatatgcttagcctatgtatgggacacattcgctaggtccaccagtatcctgtacgcaaacgcaacatcgagaagcttccgtttccaatattctatactataattacaaatgttaaaaattgtttataaaatttgtaatttgacttgtagacatcattagggtaaagatatatcaagaaattacttttttcataggaataaaaatagtcaactctcttaaataagggacaaatacaatcctctaatgacctggaaaacaactatgcaatcaacattaacaaatgtaattagaagtacaacacaatcaattttggttaaaaaattgaaagtatataaaaataaaaaaataaaaaacgtgtcttagttgataaataactcattaatcacgcctctatctctctgttttaagagtttaacagttaaagatcttcttctctatcttccttatttgactacatctccgtttattcttgggtctctctctatctctcttatccagatttatcatcgtcattacatataaacatcaaacttaacttttcaatattctcattcactttttcctcttgtgctctaattcttatccggatccgatttctctcatacaaagattctgtttttcttcttgggttattgttcttgctctgtttccgtatctcaaagtttcagctgctgaattgaatttgcattgatcgtag

SEQ ID NO: 18 - DNA, 1000 bp, Arabidopsis thaliana
  misc_feature (1)..(1000): Ceres PROMOTER PT0761 As Stated in Report Number 303
ataaattcattaataacttgtgaatttctgtgtctttgcttctttggagaaaaaaaaattatatgctaaatcaacgcatactagagttgggagttgggcctttaaaataataagtgggcctttaccttgtggacttaggccatatgagcccgagaaaatagattaaaacgattggtgcctttggaagcgacgacttcgtttgacttttcaacttaacgttttcgtctcaaatatagccgtagacaaaagacgattgcgtcaactaaaaggttaaaagagacagaaaattttgaacaaacgtttcaactatatgtgaacaaaattttgatttttttcccaaaattcccattgcatatggaaaaagattttgatttggaccatgattttttttttgttgaattaatgttaaatagaccatggatattgaatttcttttgtgagtgtatgtatgatatgtaaaagcttcaaattgtgaaatttacaatccgtttactcctttgatactatatataaaaacaccaagttattgcaagatggaaggattaagaatttaagataatggccggccactgacaaagaaggttaagctttcattaggacaatgaaagtttaaaagaataacaaaaaaagaaatgcgtgaataaatggagatagcagtgaaatctataaatatctgcataatggatgttcacgttatagttcaactaatgaagctaaattattatttgcgggaattataaatacttctaaataattatgttgacttgcaacttaataattaatccttcattttcacaacacatacagggaactttgcttggccacccagacatattattatttcaataattattcaattgctaaaaaatattagaaactcaacgggcatatatgaagtatggagcctcagtttctatataaagcaactctgccaagtgcattcctcatatacaaaaggaagaaacacttaaaagaaaatatcaaatctaaagtcgatcac

SEQ ID NO: 19 - DNA, 1000 bp, Arabidopsis thaliana
  misc_feature (1)..(1000): Ceres PROMOTER PT1016 As Stated in Report Number 323
aaagcactggggtcgaaaccggtagtaaactagaaatgagagttacctacttaaattgctctcctcatatttccaaagatgcaatcatccaaaaatgttagtcaacggtgaaaacaaaaagaaatggatgatgcttatgcgaatttaaaaggaaataatagttatattatatggataatataattaggttttgaatgtgatcttaattttgttttgtgatacacagccccagttcagaaaatattaagaatttgccactgaacatacgtatcccttccaaatcactgttgacatactaacatcattttaaaaatattcaataatatcttaatataaattttatgaattaattaaatcgaacaaagcctttttttacccaagataaacaaacatttggatagcttgtaagttaattatagtagttttaggcactgtaagtatcccgcataattcatatattattagtgatttggaaatttagcaaagggagtaaattataaaaaaaaaactagaatttacacaaattttgataaaagtcatgtcaattttttattttttttaaaggagatgttaaacatcactgttcctaaaaaaactttttaaaatatacggaaagacaatggagaactgtaccaaaatacacacattttggagaaaaggaaaaaaaaaaaagcaaaagaacacacacattttgtcagctgatatatatggatcgatatttctttgaatgcaataagcaataaatgtgcctaacatattctttcattatttttttttaatattctccctactaacactattgcttttctctcctttcttacgataaagatataataatgttttcacaaaatctttttacaaaaagaaaagaaaaaaacacattgaaatcttcttcctataaacgtctcttctctctttcatcactgcaagaaacttaatcacaactctctctttaatctctttgtctctctctctctctctctatctctctctcatttta

SEQ ID NO: 20 - DNA, 1000 bp, Arabidopsis thaliana
  misc_feature (1)..(1000): Ceres PROMOTER PT1026 As Stated in Report Number 324
ccaaactgatcatttgtataacctttcttgaaaatcatagatctatcttaaaactaaactgtaagctttttttttacttttcaattccctaagattatgtctccactttaatattttaccatttctcttaaacagaaaaattgaatatacctatttataagtattcaaaatttatttttaggtagaatttagcgcaaaagtttattaaactttacgtttacaatctcattatttaatattcacgtgaatgaatattttgacatggtaaatataatataacactataaataatatatttacatatgatgaaaagaagaataatttgataaattagaaattcacataatacatgaaataatcacatattaattccaccctaactagctaaatgataaaaataaaatcatataaatcagttcataaaacaaaaaatgtctcgggaaaaaaaaaacactcttccaaaagagacatgtaatagtcaatttatttctattttcataatcatcgttaatcctcattgcatgtggtacaattattctaataagaaaagtgaatcgtcccaataaataatcctaaaatgccgtcttaaaaaaggcaatctataaaattatttataaatatctaagaatgagttgtctactttccattgattaaaaagagaaaatatcataaacaaaaaaaaaaagtcagagaagttaaaccaaaagcaacaaaaaagaaaaaaagaaagaataatcaagagagaggagaagaggacggtggaagatattttgcagttgaagaaaatatctttgatattttttgtgtttgaaatatctaaaaccaaaaagtttcatctcttcattccgccaaattgactggtgatgacgtggcaactttacccacgtgtaaaagcgtctacaactgtatcgtttcttttgatattttgtttctctctttctctctgatatttttcattttcttcttcttctctctctctctccacaaagataagccaaca

SEQ ID NO: 21 - DNA, 250 bp, Arabidopsis thaliana
  misc_feature (1)..(250): Ceres PROMOTER YP2532 As Stated in Report Number 519
ttgtacggagtattttaatagctcaacaagttctgacaatccttatagaacatggccgatcggatctataggttgattgattgcttttactttgagattatttagagatttatataatgcgacttgcaatagtacttattgacttacattcaaaaaatttgactttctaagccaaagacattactatatgtaaaagcttcagatcgtgaatcttgattaaaattatctgagtagcaagaattcaaaagaa

SEQ ID NO: 22 - DNA, 2378 bp, Arabidopsis thaliana
  misc_feature (1)..(2378): Ceres PROMOTER YP2573 As Stated in Report Number 696
gcatactctttgatattgacatctaaagtttgtactttgaagcaacaaaaacgctctattttgcctttaccatgcgtaacagacttcgtctttaacttatatttgtttatatatttatttctgattgacacaaatcaaacatgtgctatttcataaagtcatttaaagttagctatttgttagatatctaattaagcacaatgaaatgaaatacaaatacaaatgcgtataatcaaacgctgacgtagaggaaatgactttaactatcgtaatagtaggtgttggtaaacaacggtgaacgaactataacttgatagttacaaatgatatatttaaaagtagatatcatagtgtttgtgataaaacttttttcaaaatgtaattcaaacaatttgaattgagtaggttgttgcttgaagtaggcgttcctcattatacgaatcttttgcgtttctctttgttttcatgccacgtgttgtctctaccgctcacacgccacaagaacattcttgaaaaggcttatcaaacaactttaagaacatatttctattctacccaacagtaagaaaaataaaatatatcagttatttggtttgacatagctagcgtccaactaattagaaatgatcagtttatttacattatcatccaagaaaatgcattgattcataatgattaatacaaattttcattggttattcatgggtacttttcttaccattaatgctaaaaaataaaataaaattcaccaatgtacaataagtttcttaattagcacacttttttgcgtttggtcatctgttttatgtttggttttataagtatagcattacaattattattttagctttaaaaacttaaaatgtaaattttaaacacgtgaatgtgcaagttattatgcatgtggttaataataaagggtacatgtgcgtatgagctgtgaatgtgcgtatatggcagtggacagccgtaaatgtctgaccaaaatcgttttccttatctttttacccaacagtactactactgccttaggttaaaactcatcttgtcggacgcttcttcttacaagtttacgtcttttatattctaaaattgtaacaatatattaagtttgagaaatctttcttattaattattactacgtcatttttatgtgttaactatttttgtcgaagacttttttattcttgatcaattagtccgaaaatcactactaattcgtaaacaaatttttattattaaatacctctctgtttttcctcatttgagaacgtgatataaaatcaacataatcgaatcttcacagttcacactagtttatcattataataagtgaggggattacattttaggtatttagagaaaacggggaaaagataccaaaatatcttagagacccgtggttctatatgtacattttaatttttaatttgctaaagtcagccatgaatactatagtatacctagtcagatttgatgatgatggttgttaaacccaccgctcctcctagtcaaacatcttctacaatttccaaacacattcaaaaaacgcagaaaaaaaaaaaaaaaatgtgtatatgtgttacaattcttaaaagaaaatgtaaagccagataattgttacaagagatgtacaaagctccaagtgtgaagaacatcaacatgaatatgatccatgaaatctaccaaatttccgtgcttgcactttcccatacatttttgtttttcacctaactcttacgaacctaaaataaaataaaatacaaaaaaagggagaaagatttgagcacttgtcgacacatcgtagcatcctacctctgccgacacgtggctcgagacaaatacggaactcccggtcggaacagaaacaaatacttattttgactacctgaaattcgacacgtcatcaccttctagactcaaaagagtacaaaggccaatgagactctaccattcctaacgaagtgtcacgtcacttcctatcaaacctttccttaaatacgttcactgccgcgtcactcctcttttttggtcacgtttcctatttttgtactaatacttcttcttctctctacacatattttgttggtaatcacatcgaagattcgaaattggatcacatttggtaatcaacttttttttccaaagtctccacgtgtatggacgattacatacgtacacttatttattttcgctttggtggataaactcgatgttaccaaaagtatacactatctaattaccgaaccaaaattaatctgaaacccttctatatatatatactttctttgcaataacagagtgaaagcaaacattcaaaaaaaaaaaaaaaccattttttttttcttactcaaatcttttttagtatcattctccaa

(19) United States
(12) Patent Application Publication
(10) Pub. No.: US 2010/0170002 A1
(43) Pub. Date: Jul. 1, 2010

(54) PROMOTER, PROMOTER CONTROL ELEMENTS, AND COMBINATIONS, AND USES THEREOF

(75) Inventors: Leonard Medrano, Azusa, CA (US); Yiwen Fang, Los Angeles, CA (US); Richard Schneeberger, Carlsbad, CA (US); Yu-Ping Lu, Camarillo, CA (US); Nickolai Alexandrov, Thousand Oaks, CA (US); Tatiana Tatarinova, Los Angeles, CA (US); Jack Okamuro, Oak Park, CA (US)

Correspondence Address: BIRCH STEWART KOLASCH & BIRCH, PO BOX 747, FALLS CHURCH, VA, 22040-0747, US

(73) Assignee: CERES, INC., Thousand Oaks, CA (US)

(21) Appl. No.: 12/294,418

(22) PCT Filed: Mar. 23, 2007
(86) PCT No.: PCT/US2007/064848

§ 371 Date: Feb. 1, 2010

Related U.S. Application Data
(60) Provisional application No. 60/785,794, filed on Mar. 24, 2006.

(51) Int. Cl. A01H 1/00; C12N 15/82; C12N 5/10; C12N 15/63; C07H 21/04; A01H 5/00

(52) U.S. Cl. 800/278; 435/468; 435/419; 435/320.1; 536/23.6; 800/298

(57) ABSTRACT

The present invention is directed to promoter sequences and promoter control elements, polynucleotide constructs comprising the promoters and control elements, and methods of identifying the promoters, control elements, or fragments thereof. The invention further relates to the use of the present promoters or promoter control elements to modulate transcript levels in plants, and plants containing such promoters or promoter control elements.

This application claims priority to U.S. Provisional Application No. 60/785,794 filed Mar. 24, 2006, the entire contents of which are hereby incorporated by reference.

### FIELD OF THE INVENTION

The present invention relates to promoters and promoter control elements that are useful for modulating transcription of a desired polynucleotide. Such promoters and promoter control elements can be included in polynucleotide constructs, expression cassettes, vectors, or inserted into the chromosome or as an exogenous element, to modulate in vivo and in vitro transcription of a polynucleotide. Host cells, including plant cells, and organisms, such as regenerated plants therefrom, with desired traits or characteristics using polynucleotides comprising the promoters and promoter control elements of the present invention are also a part of the invention.

### BACKGROUND OF THE INVENTION

This invention relates to promoter sequences and promoter control element sequences which are useful for the transcription of polynucleotides in a host cell or transformed host organism.

The introduction of genes into plants has resulted in the development of plants having new and useful phenotypes such as pathogen resistance, higher levels of healthier types of oils, novel production of healthful components such as beta-carotene synthesis in rice. An introduced gene is generally a chimeric gene composed of the coding region that confers the desired trait and regulatory sequences. One regulatory sequence is the promoter, which is located 5′ to the coding region. This sequence is involved in regulating the pattern of expression of a coding region 3′ thereof. The promoter sequence binds RNA polymerase complex as well as one or more transcription factors that are involved in producing the RNA transcript of the coding region.

The promoter region of a gene used in plant transformation is most often derived from a different source than is the coding region. It may be from a different gene of the same species of plant, from a different species of plant, from a plant virus, or it may be a composite of different natural and/or synthetic sequences. Properties of the promoter sequence generally determine the pattern of expression for the coding region that is operably linked to the promoter. Promoters with different characteristics of expression have been described. The promoter may confer broad expression as in the case of the widely-used cauliflower mosaic virus (CaMV) 35S promoter. The promoter may confer tissue-specific expression as in the case of the seed-specific phaseolin promoter. The promoter may confer a pattern for developmental changes in expression. The promoter may be induced by an applied chemical compound, or by an environmental condition applied to the plant.

The promoter that is used to regulate a particular coding region is determined by the desired expression pattern for that coding region, which itself is determined by the desired resulting phenotype in the plant. For example, herbicide resistance is desired throughout the plant so the 35S promoter is appropriate for expression of an herbicide-resistance gene. A seed-specific promoter is appropriate for changing the oil content of soybean seed. An endosperm-specific promoter is appropriate for changing the starch composition of corn seed. A root-specific promoter can be important for improving water or nutrient up-take in a plant. Control of expression of an introduced gene by the promoter is important because it is sometimes detrimental to have expression of an introduced gene in non-target tissues.

One of the primary goals of biotechnology is to obtain organisms, such as plants, mammals, yeast, and prokaryotes having particular desired characteristics or traits. Examples of these characteristics or traits abound and may include, for example, in plants, virus resistance, insect resistance, herbicide resistance, enhanced stability or additional nutritional value. Recent advances in genetic engineering have enabled researchers in the field to incorporate polynucleotide sequences into host cells to obtain the desired qualities in the organism of choice. This technology permits one or more polynucleotides from a source different than the organism of choice to be transcribed by the organism of choice. If desired, the transcription and/or translation of these new polynucleotides can be modulated in the organism to exhibit a desired characteristic or trait. Alternatively, new patterns of transcription and/or translation of polynucleotides endogenous to the organism can be produced.

### SUMMARY OF THE INVENTION

The present invention is directed to isolated polynucleotide sequences that comprise promoters and promoter control elements from plants, especially Arabidopsis thaliana, and other promoters and promoter control elements functional in plants.

It is an object of the present invention to provide isolated polynucleotides that are promoter or promoter control sequences. These promoter sequences comprise, for example,
(1) a polynucleotide having a nucleotide sequence according to SEQ. ID. Nos. 1-22;
(2) a polynucleotide having a nucleotide sequence having at least 80% sequence identity to a sequence according to SEQ. ID. Nos. 1-22; and
(3) a polynucleotide having a nucleotide sequence which hybridizes to a sequence according to SEQ. ID. Nos. 1-22 under a condition establishing a Tm-5° C.

Promoter or promoter control element sequences of the present invention are capable of modulating preferential transcription.

In another embodiment, the present promoter control elements are capable of serving as or fulfilling the function, for example, as a core promoter, a TATA box, a polymerase binding site, an initiator site, a transcription binding site, an enhancer, an inverted repeat, a locus control region, or a scaffold/matrix attachment region.

It is yet another object of the present invention to provide a polynucleotide that includes at least a first and a second promoter control element. The first promoter control element is a promoter control element sequence as discussed above, and the second promoter control element is heterologous to the first control element; wherein, the first and second control elements are operably linked. Such promoters may modulate transcript levels preferentially in a particular tissue or under particular conditions.

In another embodiment, the present isolated polynucleotide comprises a promoter or a promoter control element as described above, wherein the promoter or promoter control element is operably linked to a polynucleotide to be transcribed.

In another embodiment of the present invention, the promoter and promoter control elements of the instant invention are operably linked to a heterologous polynucleotide that is a regulatory sequence.

It is another object of the present invention to provide a host cell comprising an isolated polynucleotide or vector as described above or fragment thereof. Host cells include, for instance, bacterial, yeast, insect, mammalian, and plant. The host cell can comprise a promoter or promoter control element exogenous to the genome. Such a promoter can modulate transcription in cis- and in trans-.

In yet another embodiment, the host cell is a plant cell capable of regenerating into a plant.

It is yet another embodiment of the present invention to provide a plant comprising an isolated polynucleotide or vector described above.

It is another object of the present invention to provide a method of modulating transcription in a sample that contains either a cell-free system of transcription or host cell. This method comprises providing a polynucleotide or vector according to the present invention as described above, and contacting the sample of the polynucleotide or vector with conditions that permit transcription.

In another embodiment of the present method, the polynucleotide or vector preferentially modulates, depending upon the function of the particular promoter, constitutive transcription, stress induced transcription, light induced transcription, dark induced transcription, leaf transcription, root transcription, stem or shoot transcription, silique transcription, callus transcription, flower transcription, immature bud and inflorescence specific transcription, senescing induced transcription, germination transcription or drought transcription.

Other and further objects of the present invention will be made clear or become apparent from the following description.

### BRIEF DESCRIPTION OF THE TABLES AND FIGURES

Table 1 consists of the Expression Reports for each promoter of the invention providing the nucleotide sequence for each promoter and details for expression driven by each of the nucleic acid promoter sequences as observed in transgenic plants. The results are presented as summaries of the spatial expression, which provides information as to gross and/or specific expression in various plant organs and tissues. The observed expression pattern is also presented, which gives details of expression during different generations or different developmental stages within a generation. Additional information is provided regarding the source organism of the promoter, and the vector and marker genes used for the construct. The following symbols are used consistently throughout the Table:

T1: First generation transformant

T2: Second generation transformant

T3: Third generation transformant

(L): low expression level

(M): medium expression level

(H): high expression level

Each row of the table begins with heading of the data to be found in the section. The following provides a description of the data to be found in each section:

| | Heading in Table 1 | Description |
|---|---|---|
| 1. | Promoter Expression Report # | Identifies the particular promoter by its |
| | | construct ID. |
| 2. | Promoter tested in: | Identifies the organism in which the promoter- |
| | | marker vector was tested. |
| 3. | Spatial expression summary: | Identifies the specific parts of the plant where |
| | | various levels of GFP expression are |
| | | observed. Expression levels are noted as |
| | | either low (L), medium (M), or high (H). |
| 4. | Observed expression pattern: | Provides a general explanation of where GFP |
| | | expression in different generations of plants |
| | | was observed. |
| 5. | Source promoter organism: | Identifies the plant species from which the |
| | | promoter was derived. |
| 6. | Vector: | Identifies the vector used into which a |
| | | promoter was cloned. |
| 7. | Marker type: | Identifies the type of marker linked to the |
| | | promoter. The marker is used to determine |
| | | patterns of gene expression in plant tissue. |
| 8. | Generation screened: □T1 Mature | Identifies the plant generation(s) used in the |
| | □T2 Seedling □T2 Mature □T3 | screening process. T1 plants are those plants |
| | Seedling | subjected to the transformation event while |
| | | the T2 generation plants are from the seeds |
| | | collected from the T1 plants and T3 plants are |
| | | from the seeds of T2 plants. |
| 9. | Inductions completed: | Provides summary of experiment schedule. |
| 10. | Table 1 - T1 Mature Plant Expression: | Identifies plant tissues that were observed for |
| | | possible expression, and identifies (H, M or L) |
| | | level of observed expression. |
| 11. | Table 2 - T2 Seedling Expression: | Identifies plant tissues that were observed for |
| | | possible expression, and identifies (H, M or L) |
| | | level of observed expression. |
| 12. | Table 3 - T2 Mature Plant Expression: | Identifies plant tissues that were observed for |
| | | possible expression, and identifies (H, M or L) |
| | | level of observed expression. |
| 13. | Table 4 - Utility | Provides a description of the utility of the |
| | | sequence. |
| 14. | Promoter Candidate ID: | Provides an internal ID number for the |
| | | promoter. |
| 15. | Construct | Identifies the promoter by its construct ID and |
| | Promoter Candidate I.D. | internal candidate number |
| 16. | Lines/Events expressing: | Identifies the line/event numbers that |
| | | expressed under the promoter. |

FIG. 1—pNewbin4-HAP1-GFP

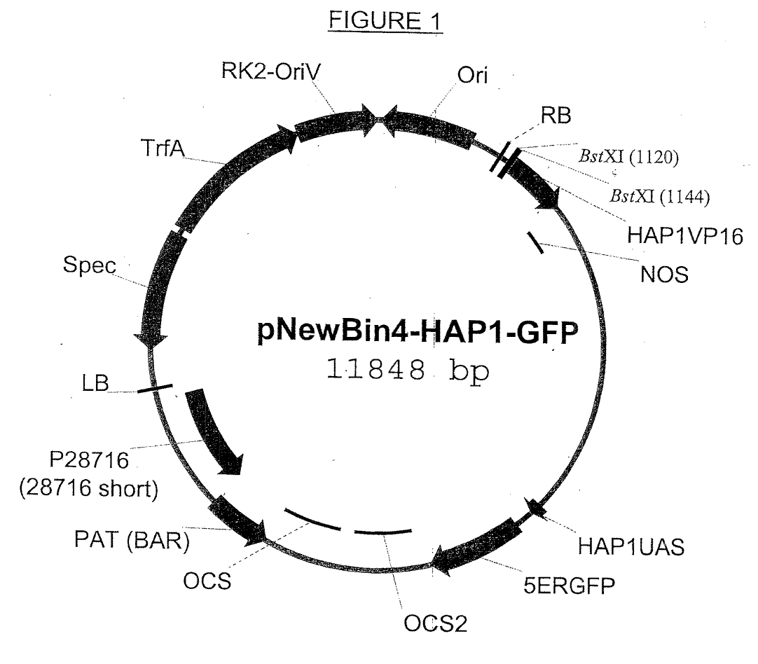

FIG. 1 is a schematic representation of a vector that is useful to insert promoters of the invention into a plant. The definitions of the abbreviations used in the vector map are as follows:
Ori—the origin of replication used by an E. coli host
RB—sequence for the right border of the T-DNA from pMOG800
BstXI—restriction enzyme cleavage site used for cloning
HAP1VP16—coding sequence for a fusion protein of the HAP1 and VP16 activation domains
NOS—terminator region from the nopaline synthase gene
HAP1UAS—the upstream activating sequence for HAP1
5ERGFP—the green fluorescent protein gene that has been optimized for localization to the endoplasmic reticulum
OCS2—the terminator sequence from the octopine synthase 2 gene
OCS—the terminator sequence from the octopine synthase gene
p28716 (a.k.a 28716 short)—promoter used to drive expression of the PAT (BAR) gene
PAT (BAR)—a marker gene conferring herbicide resistance
LB—sequence for the left border of the T-DNA from pMOG800
Spec—a marker gene conferring spectinomycin resistance
TrfA—transcription repression factor gene
RK2-OriV—origin of replication for Agrobacterium

### DETAILED DESCRIPTION OF THE INVENTION

The following definitions and methods are provided to better define the present invention and to guide those of ordinary skill in the art in the practice of the present invention. Unless otherwise noted, terms are to be understood according to conventional usage by those of ordinary skill in the relevant art.

The invention disclosed herein provides promoters capable of driving the expression of an operably linked transgene. The design, construction, and use of these promoters is one object of this invention. The promoter sequences, SEQ ID NOs: 1-22, are capable of transcribing operably linked nucleic acid molecules in particular plant tissues/organs or during particular plant growth stages, and therefore can selectively regulate to expression of transgenes in these tissues/organs or at these times of plant development.

### 1. DEFINITIONS

Chimeric: The term “chimeric” is used to describe polynucleotides or genes, or constructs wherein at least two of the elements of the polynucleotide or gene or construct, such as the promoter and the polynucleotide to be transcribed and/or other regulatory sequences and/or filler sequences and/or complements thereof, are heterologous to each other.

Broadly Expressing Promoter: Promoters referred to herein as “broadly expressing promoters” actively promote transcription under most, but not necessarily all, environmental conditions and states of development or cell differentiation. Examples of broadly expressing promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. A similar analysis can be applied to polynucleotides. Generally, each domain has been associated with either a conserved primary sequence or a sequence motif. Generally these conserved primary sequence motifs have been correlated with specific in vitro and/or in vivo activities. A domain can be any length, including the entirety of the polynucleotide to be transcribed. Examples of domains include, without limitation, AP2, helicase, homeobox, zinc finger, etc.

Endogenous: The term “endogenous,” within the context of the current invention refers to any polynucleotide, polypeptide or protein sequence which is a natural part of a cell or organism(s) regenerated from said cell. In the context of promoter, the term “endogenous coding region” or “endogenous cDNA” refers to the coding region that is naturally operably linked to the promoter.

Enhancer/Suppressor: An “enhancer” is a DNA regulatory element that can increase the steady state level of a transcript, usually by increasing the rate of transcription initiation. Enhancers usually exert their effect regardless of the distance, upstream or downstream location, or orientation of the enhancer relative to the start site of transcription. In contrast, a “suppressor” is a corresponding DNA regulatory element that decreases the steady state level of a transcript, again usually by affecting the rate of transcription initiation. The essential activity of enhancer and suppressor elements is to bind a protein factor(s). Such binding can be assayed, for example, by methods described below. The binding is typically in a manner that influences the steady state level of a transcript in a cell or in an in vitro transcription extract.

Exogenous: As referred to within, “exogenous” is any polynucleotide, polypeptide or protein sequence, whether chimeric or not, that is introduced into the genome of a host cell or organism regenerated from said host cell by any means other than by a sexual cross. Examples of means by which this can be accomplished are described below, and include Agrobacterium-mediated transformation (of dicots—e.g. Salomon et al. (1984) EMBO J. 3:141; Herrera-Estrella et al. (1983) EMBO J. 2:987; of monocots, representative papers are those by Escudero et al. (1996) Plant J. 10:355), Ishida et al. (1996) Nature Biotech 14:745, May et al. (1995) Bio/Technology 13:486), biolistic methods (Armaleo et al. (1990) Current Genetics 17:97), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Homologous: In the current invention, a “homologous” polynucleotide refers to a polynucleotide that shares sequence similarity with the polynucleotide of interest. This similarity may be in only a fragment of the sequence and often represents a functional domain such as, examples including without limitation a DNA binding domain or a domain with tyrosine kinase activity. The functional activities of homologous polynucleotides are not necessarily the same.

Inducible Promoter: An “inducible promoter” in the context of the current invention refers to a promoter, the activity of which is influenced by certain conditions, such as light, temperature, chemical concentration, protein concentration, conditions in an organism, cell, or organelle, etc. A typical example of an inducible promoter, which can be utilized with the polynucleotides of the present invention, is PARSK1, the promoter from an Arabidopsis gene encoding a serine-threonine kinase enzyme, and which promoter is induced by dehydration, abscissic acid and sodium chloride (Wang and Goodman (1995) Plant J. 8:37). Examples of environmental conditions that may affect transcription by inducible promoters include anaerobic conditions, elevated temperature, the presence or absence of a nutrient or other chemical compound or the presence of light.

Misexpression: The term “misexpression” refers to an increase or a decrease in the transcription of a coding region into a complementary RNA sequence as compared to the wild-type. This term also encompasses expression and/or translation of a gene or coding region or inhibition of such transcription and/or translation for a different time period as compared to the wild-type and/or from a non-natural location within the plant genome, including a gene or coding region from a different plant species or from a non-plant organism.

Modulate Transcription Level: As used herein, the phrase “modulate transcription” describes the biological activity of a promoter sequence or promoter control element. Such modulation includes, without limitation, up- and down-regulation of initiation of transcription, rate of transcription, and/or transcription levels.

Operable Linkage: An “operable linkage” is a linkage in which a promoter sequence or promoter control element is connected to a polynucleotide sequence (or sequences) in such a way as to place transcription of the polynucleotide sequence under the influence or control of the promoter or promoter control element. Two DNA sequences (such as a polynucleotide to be transcribed and a promoter sequence linked to the 5′ end of the polynucleotide to be transcribed) are said to be operably linked if induction of promoter function results in the transcription of mRNA encoding the polynucleotide and if the nature of the linkage between the two DNA sequences does not (1) result in the introduction of a frame-shift mutation, (2) interfere with the ability of the promoter sequence to direct the expression of the protein, antisense RNA, RNAi or ribozyme, or (3) interfere with the ability of the DNA template to be transcribed. Thus, a promoter sequence would be operably linked to a polynucleotide sequence if the promoter was capable of effecting transcription of that polynucleotide sequence.

Percentage of sequence identity As used herein, the term “percent sequence identity” refers to the degree of identity between any given query sequence and a subject sequence. A subject sequence typically has a length that is from about 80 percent to 250 percent of the length of the query sequence, e.g., 82, 85, 87, 89, 90, 93, 95, 97, 99, 100, 105, 110, 115, or 120, 130, 140, 150, 160, 170, 180, 190, 200, 210, 220, 230, 240, or 250 percent of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al. (2003) Nucleic Acids Res. 31(13):3497-500.

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For an alignment of multiple nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher website and at the European Bioinformatics Institute website on the World Wide Web.

To determine a percent identity for polypeptide or nucleic acid sequences between a query and a subject sequence, the sequences are aligned using Clustal W and the number of identical matches in the alignment is divided by the query length, and the result is multiplied by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Plant Promoter: A “plant promoter” is a promoter capable of initiating transcription in plant cells and can modulate transcription of a polynucleotide. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-1 (ubi-1) promoter known to those of skill in the art.

Plant Tissue: The term “plant tissue” includes differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

Preferential Transcription: “Preferential transcription” is defined as transcription that occurs in a particular pattern of cell types or developmental times or in response to specific stimuli or combination thereof. Non-limitive examples of preferential transcription include: high transcript levels of a desired sequence in root tissues; detectable transcript levels of a desired sequence in certain cell types during embryogenesis; and low transcript levels of a desired sequence under drought conditions. Such preferential transcription can be determined by measuring initiation, rate, and/or levels of transcription.

Promoter: A “promoter” is a DNA sequence that directs the transcription of a polynucleotide. Typically a promoter is located in the 5′ region of a polynucleotide to be transcribed, proximal to the transcriptional start site of such polynucleotide. More typically, promoters are defined as the region upstream of the first exon; more typically, as a region upstream of the first of multiple transcription start sites; more typically, as the region downstream of the preceding gene and upstream of the first of multiple transcription start sites; more typically, the region downstream of the polyA signal and upstream of the first of multiple transcription start sites; even more typically, about 3,000 nucleotides upstream of the ATG of the first exon; even more typically, 2,000 nucleotides upstream of the first of multiple transcription start sites. The promoters of the invention comprise at least a core promoter as defined above. Frequently promoters are capable of directing transcription of genes located on each of the complementary DNA strands that are 3′ to the promoter. Stated differently, many promoters exhibit bidirectionality and can direct transcription of a downstream gene when present in either orientation (i.e. 5′ to 3′ or 3′ to 5′ relative to the coding region of the gene). Additionally, the promoter may also include at least one control element such as an upstream element. Such elements include UARs and optionally, other DNA sequences that affect transcription of a polynucleotide such as a synthetic upstream element.

Promoter Control Element: The term “promoter control element” as used herein describes elements that influence the activity of the promoter. Promoter control elements include transcriptional regulatory sequence determinants such as, but not limited to, enhancers, scaffold/matrix attachment regions, TATA boxes, transcription start locus control regions, UARs, URRs, other transcription factor binding sites and inverted repeats.

Public sequence: The term “public sequence,” as used in the context of the instant application, refers to any sequence that has been deposited in a publicly accessible database prior to the filing date of the present application. This term encompasses both amino acid and nucleotide sequences. Such sequences are publicly accessible, for example, on the BLAST databases on the NCBI FTP web site (accessible via the internet). The database at the NCBI FTP site utilizes “gi” numbers assigned by NCBI as a unique identifier for each sequence in the databases, thereby providing a non-redundant database for sequence from various databases, including GenBank, EMBL, DBBJ (DNA Database of Japan) and PDB (Brookhaven Protein Data Bank).

Regulatory Regions: The term “regulatory region” refers to nucleotide sequences that, when operably linked to a sequence, influence transcription initiation or translation initiation or transcription termination of said sequence and the rate of said processes, and/or stability and/or mobility of a transcription or translation product. As used herein, the term “operably linked” refers to positioning of a regulatory region and said sequence to enable said influence. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Regulatory regions can be classified in two categories, promoters and other regulatory regions.

Regulatory Sequence: The term “regulatory sequence,” as used in the current invention, refers to any nucleotide sequence that influences transcription or translation initiation and rate, or stability and/or mobility of a transcript or polypeptide product.

Regulatory sequences include, but are not limited to, promoters, promoter control elements, protein binding sequences, 5′ and 3′ UTRs, transcriptional start sites, termination sequences, polyadenylation sequences, introns, certain sequences within amino acid coding sequences such as secretory signals, protease cleavage sites, etc.

Specific Promoters: In the context of the current invention, “specific promoters” refers to a subset of promoters that have a high preference for modulating transcript levels in a specific tissue or organ or cell and/or at a specific time during development of an organism. By “high preference” is meant at least 3-fold, preferably 5-fold, more preferably at least 10-fold still more preferably at least 20-fold, 50-fold or 100-fold increase in transcript levels under the specific condition over the transcription under any other reference condition considered. Typical examples of temporal and/or tissue or organ specific promoters of plant origin that can be used with the polynucleotides of the present invention, are: PTA29, a promoter which is capable of driving gene transcription specifically in tapetum and only during anther development (Koltonow et al. (1990) Plant Cell 2:1201; RCc2 and RCc3, promoters that direct root-specific gene transcription in rice (Xu et al. (1995) Plant Mol. Biol. 27:237; TobRB27, a root-specific promoter from tobacco (Yamamoto et al. (1991) Plant Cell 3:371). Examples of tissue-specific promoters under developmental control include promoters that initiate transcription only in certain tissues or organs, such as root, ovule, fruit, seeds, or flowers. Other specific promoters include those from genes encoding seed storage proteins or the lipid body membrane protein, oleosin. A few root-specific promoters are noted above. See also “Preferential transcription.”

Stringency: “Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship between hybridization conditions and Tₘ (in ° C.) is expressed in the mathematical equation:

Tₘ=81.5−16.6(log₁₀[Na⁺])+0.41(% G+C)−(600/N) (I)

where N is the number of nucleotides of the nucleic acid molecule probe. This equation works well for probes 14 to 70 nucleotides in length that are identical to the target sequence. The equation below, for Tₘ, of DNA-DNA hybrids, is useful for probes having lengths in the range of 50 to greater than 500 nucleotides, and for conditions that include an organic solvent (formamide):

Tₘ=81.5+16.6 log {[Na⁺]/(1+0.7[Na⁺])}+0.41(% G+C)−500/L0.63(% formamide) (II)

where L represents the number of nucleotides in the probe in the hybrid (21). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (Frischauf et al. (1983) J. Mol Biol, 170: 827-842), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

T₀: The term “T₀” refers to the whole plant, explant or callus tissue, inoculated with the transformation medium.

T₁: The term T₁ refers to either the progeny of the T₀ plant, in the case of whole-plant transformation, or the regenerated seedling in the case of explant or callous tissue transformation.

T₂: The term T₂ refers to the progeny of the T₁ plant. T₂ progeny are the result of self-fertilization or cross-pollination of a T₁ plant.

T₃: The term T₃ refers to second generation progeny of the plant that is the direct result of a transformation experiment. T₃ progeny are the result of self-fertilization or cross-pollination of a T₂ plant.

TATA to start: “TATA to start” shall mean the distance, in number of nucleotides, between the primary TATA motif and the start of transcription.

Transgenic plant: A “transgenic plant” is a plant having one or more plant cells that contain at least one exogenous polynucleotide introduced by recombinant nucleic acid methods.

Translational start site: In the context of the present invention, a “translational start site” is usually an ATG or AUG in a transcript, often the first ATG or AUG. A single protein encoding transcript, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single polynucleotide to be transcribed may have multiple transcriptional start sites, some of which may be specific for transcription in a particular cell-type or tissue or organ. “+1” is stated relative to the transcription start site and indicates the first nucleotide in a transcript.

Upstream Activating Region (UAR): An “Upstream Activating Region” or “UAR” is a position or orientation dependent nucleic acid element that primarily directs tissue, organ, cell type, or environmental regulation of transcript level, usually by affecting the rate of transcription initiation. Corresponding DNA elements that have a transcription inhibitory effect are called herein “Upstream Repressor Regions” or “URR”s. The essential activity of these elements is to bind a protein factor. Such binding can be assayed by methods described below. The binding is typically in a manner that influences the steady state level of a transcript in a cell or in vitro transcription extract.

Untranslated region (UTR): A “UTR” is any contiguous series of nucleotide bases that is transcribed, but is not translated. A 5′ UTR lies between the start site of the transcript and the translation initiation codon and includes the +1 nucleotide. A 3′ UTR lies between the translation termination codon and the end of the transcript. UTRs can have particular functions such as increasing mRNA message stability or translation attenuation. Examples of 3′ UTRs include, but are not limited to polyadenylation signals and transcription termination sequences.

### 2. USE OF THE PROMOTERS OF THE INVENTION

The promoters and promoter control elements of this invention are capable of modulating transcription. Such promoters and promoter control elements can be used in combination with native or heterologous promoter fragments, control elements or other regulatory sequences to modulate transcription and/or translation.

Specifically, promoters and control elements of the invention can be used to modulate transcription of a desired polynucleotide, which includes without limitation:
a) antisense;
b) ribozymes;
c) coding sequences; or
d) fragments thereof.

The promoter also can modulate transcription in a host genome in cis- or in trans-.

In an organism, such as a plant, the promoters and promoter control elements of the instant invention are useful to produce preferential transcription which results in a desired pattern of transcript levels in a particular cells, tissues, or organs, or under particular conditions.

### 4. IDENTIFYING AND ISOLATING PROMOTER SEQUENCES OF THE INVENTION

The promoters and promoter control elements of the present invention are presented in the Promoter Reports of Table 1 and were identified from Arabidopsis thaliana. Isolation from genomic libraries of polynucleotides comprising the sequences of the promoters and promoter control elements of the present invention is possible using known techniques. For example, polymerase chain reaction (PCR) can amplify the desired polynucleotides utilizing primers designed from SEQ ID NOs: 1-22. Polynucleotide libraries comprising genomic sequences can be constructed according to Sambrook et al., Molecular Cloning: A Laboratory Manual, 2″ Ed. (1989) Cold Spring Harbor Press, Cold Spring Harbor, N.Y.), for example.

Other procedures for isolating polynucleotides comprising the promoter sequences of the invention include, without limitation, tail-PCR, and 5′ rapid amplification of cDNA ends (RACE). See, for tail-PCR, for example, Liu et al. (1995) Plant J 8(3): 457-463; Liu et al. (1995) Genomics 25: 674-681; Liu et al. (1993) Nucl. Acids Res. 21(14): 3333-3334; and Zoe et al. (1999) BioTechniques 27(2): 240-248; for RACE, see, for example, PCR Protocols: A Guide to Methods and Applications, (1990) Academic Press, Inc.

In addition, the promoters and promoter control elements described in the Promoter Reports in Table 1 (SEQ. ID. Nos. 1-22) can be chemically synthesized according to techniques in common use. See, for example, Beaucage et al. (1981) Tet. Lett. 22: 1859 and U.S. Pat. No. 4,668,777. Such chemical oligonucleotide synthesis can be carried out using commercially available devices, such as, Biosearch 4600 or 8600 DNA synthesizer, by to Applied Biosystems, a division of Perkin-Elmer Corp., Foster City, Calif., USA; and Expedite by Perceptive Biosystems, Framingham, Mass., USA.

Included in the present invention are promoters exhibiting nucleotide sequence identity to SEQ. ID. Nos. 1-22 namely that exhibits at least 80% sequence identity, at least 85%, at least 90%, and at least 95%, 96%, 97%, 98% or 99% sequence identity compared to SEQ. ID. Nos. 1-22. Such sequence identity can be calculated by the algorithms and computers programs described above.

### 5. TESTING OF PROMOTERS

Promoters of the invention were tested for activity by cloning the sequence into an appropriate vector, transforming plants with the construct and assaying for marker gene expression. Recombinant DNA constructs were prepared which comprise the promoter sequences of the invention inserted into a vector suitable for transformation of plant cells. The construct can be made using standard recombinant DNA techniques (Sambrook et al. 1989) and can be introduced to the species of interest by Agrobacterium-mediated transformation or by other means of transformation as referenced below.

The vector backbone can be any of those typical in the art such as plasmids, viruses, artificial chromosomes, BACs, YACs and PACs and vectors of the sort described by
(a) BAC: Shizuya et al. (1992) Proc. Natl. Acad. Sci. USA 89: 8794-8797; Hamilton et al. (1996) Proc. Natl. Acad. Sci. USA 93: 9975-9979;
(b) YAC: Burke et al. (1987) Science 236:806-812;
(c) PAC: Sternberg N. et al. (1990) Proc Natl Acad Sci USA. 87(1):103-7;
(d) Bacteria-Yeast Shuttle Vectors: Bradshaw et al. (1995) Nucl Acids Res 23: 4850-4856;
(e) Lambda Phage Vectors: Replacement Vector, e.g., Frischauf et al. (1983) J Mol Biol 170: 827-842; or Insertion vector, e.g., Huynh et al. (1985) In: Glover N M (ed) DNA Cloning: A practical Approach, Vol. 1 Oxford: IRL Press; T-DNA gene fusion vectors:Walden et al. (1990) Mol Cell Biol 1: 175-194; and
(g) Plasmid vectors: Sambrook et al., infra.

Typically, the construct comprises a vector containing a promoter sequence of the present invention operationally linked to any marker gene. The promoter was identified as a promoter by the expression of the marker gene. Although many marker genes can be used, Green Fluroescent Protein (GFP) is preferred. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin. Vectors can also include origins of replication, scaffold attachment regions (SARs), markers, homologous sequences, introns, etc.

### 6. CONSTRUCTING PROMOTERS WITH CONTROL ELEMENTS

### 6.1 Combining Promoters and Promoter Control Elements

The promoter and promoter control elements of the present invention, both naturally occurring and synthetic, can be used alone or combined with each other to produce the desired preferential transcription. Also, the promoters of the invention can be combined with other known sequences to obtain other useful promoters to modulate, for example, tissue transcription specific or transcription specific to certain conditions. Such preferential transcription can be determined using the techniques or assays described above.

Promoters can contain any number of control elements. For example, a promoter can contain multiple transcription binding sites or other control elements. One element may confer tissue or organ specificity; another element may limit transcription to specific time periods, etc. Typically, promoters will contain at least a basal or core promoter as described above. Any additional element can be included as desired. For example, a fragment comprising a basal or “core” promoter can be fused with another fragment with any number of additional control elements.

The following are promoters that are induced under stress conditions and can be combined with those of the present invention: ldh1 (oxygen stress; tomato; see Germain and Ricard (1997) Plant Mol Biol 35:949-54), GPx and CAT (oxygen stress; mouse; see Franco et al. (1999) Free Radic Biol Med 27:1122-32), ci7 (cold stress; potato; see Kirch et al. (1997) Plant Mol Biol. 33:897-909), Bz2 (heavy metals; maize; see Marrs and Walbot (1997) Plant Physiol 113:93-102), HSP32 (hyperthermia; rat; see Raju and Maines (1994) Biochim Biophys Acta 1217:273-80); MAPKAPK-2 (heat shock; Drosophila; see Larochelle and Suter (1995) Gene 163:209-14).

In addition, the following examples of promoters are induced by the presence or absence of light can be used in combination with those of the present invention: Topoisomerase II (pea; see Reddy et al. (1999) Plant Mol Biol 41:125-37), chalcone synthase (soybean; see Wingender et al. (1989) Mol Gen Genet 218:315-22) mdm2 gene (human tumor; see Saucedo et al. (1998) Cell Growth Differ 9:119-30), Clock and BMAL1 (rat; see Namihira et al. (1999) Neurosci Lett 271:1-4, PHYA (Arabidopsis; see Canton and Quail (1999) Plant Physiol 121:1207-16), PRB-1b (tobacco; see Sessa et al. (1995) Plant Mol Biol 28:537-47) and Ypr10 (common bean; see Walter et al. (1996) Eur Biochem 239:281-93).

The promoters and control elements of the following genes can be used in combination with the present invention to confer tissue specificity: MipB (iceplant; Yamada et al. (1995) Plant Cell 7:1129-42) and SUCS (root nodules; broadbean; Kuster et al. (1993) Mol Plant Microbe Interact 6:507-14) for roots, OsSUTI (rice; Hirose et al. (1997) Plant Cell Physiol 38:1389-96) for leaves, Msg (soybean; Stomvik et al. (1999) Plant Mol Biol 41:217-31) for siliques, cell (Arabidopsis; Shani et al. (1997) Plant Mol Biol 34(6):837-42) and ACT11 (Arabidopsis; Huang et al. (1997) Plant Mol Biol 33:125-39) for inflorescence.

Still other promoters are affected by hormones or participate in specific physiological processes, which can be used in combination with those of present invention.

Some examples are the ACC synthase gene that is induced differently by ethylene and brassinosteroids (mung bean; Yi et al. (1999) Plant Mol Biol 41:443-54), the TAPG1 gene that is active during abscission (tomato; Kalaitzis et al. (1995) Plant Mol Biol 28:647-56), and the 1-aminocyclopropane-1-carboxylate synthase gene (carnation; Jones et al. (1995) Plant Mol Biol 28:505-12) and the CP-2/cathepsin L gene (rat; Kim and Wright (1997) Biol Reprod 57:1467-77), both active during senescence.

Spacing between control elements or the configuration or control elements can be determined or optimized to permit the desired protein-polynucleotide or polynucleotide interactions to occur.

For example, if two transcription factors bind to a promoter simultaneously or relatively close in time, the binding sites are spaced to allow each factor to bind without steric hindrance. The spacing between two such hybridizing control elements can be as small as a profile of a protein bound to a control element. In some cases, two protein binding sites can be adjacent to each other when the proteins bind at different times during the transcription process.

Further, when two control elements hybridize the spacing between such elements will be sufficient to allow the promoter polynucleotide to hairpin or loop to permit the two elements to bind. The spacing between two such hybridizing control elements can be as small as a t-RNA loop, to as large as 10 kb.

Typically, the spacing is no smaller than 5 bases; more typically, no smaller than 8; more typically, no smaller than 15 bases; more typically, no smaller than 20 bases; more typically, no smaller than 25 bases; even more typically, no smaller than 30, 35, 40 or 50 bases.

Usually, the fragment size in no larger than 5 kb bases; more usually, no larger than 2 kb; more usually, no larger than 1 kb; more usually, no larger than 800 bases; more usually, no larger than 500 bases; even more usually, no more than 250, 200, 150 or 100 bases.

Such spacing between promoter control elements can be determined using the techniques and assays described above.

### 6.2 Vectors Used to Transform Cells/Hosts

A plant transformation construct containing a promoter of the present invention may be introduced into plants by any plant transformation method. Methods and materials for transforming plants by introducing a plant expression construct into a plant genome in the practice of this invention can include any of the well-known and demonstrated methods including electroporation (U.S. Pat. No. 5,384,253); microprojectile bombardment (U.S. Pat. No. 5,015,580; U.S. Pat. No. 5,550,318; U.S. Pat. No. 5,538,880; U.S. Pat. No. 6,160,208; U.S. Pat. No. 6,399,861; and U.S. Pat. No. 6,403,865); Agrobacterium-mediated transformation (U.S. Pat. No. 5,824,877; U.S. Pat. No. 5,591,616; U.S. Pat. No. 5,981,840; and U.S. Pat. No. 6,384,301); and protoplast transformation (U.S. Pat. No. 5,508,184).

The present promoters and/or promoter control elements may be delivered to a system such as a cell by way of a vector. For the purposes of this invention, such delivery may range from simply introducing the promoter or promoter control element by itself randomly into a cell to integration of a cloning vector containing the present promoter or promoter control element. Thus, a vector need not be limited to a DNA molecule such as a plasmid, cosmid or bacterial phage that has the capability of replicating autonomously in a host cell. All other manner of delivery of the promoters and promoter control elements of the invention are envisioned. The various T-DNA vector types are a preferred vector for use with the present invention. Many useful vectors are commercially available.

It may also be useful to attach a marker sequence to the present promoter and promoter control element in order to determine activity of such sequences. Marker sequences typically include genes that provide antibiotic resistance, such as tetracycline resistance, hygromycin resistance or ampicillin resistance, or provide herbicide resistance. Specific selectable marker genes may be used to confer resistance to herbicides such as glyphosate, glufosinate or broxynil (Comai et al. (1985) Nature 317: 741-744; Gordon-Kamm et al. (1990) Plant Cell 2: 603-618; and Stalker et al. (1988) Science 242: 419-423). Other marker genes exist which provide hormone responsiveness.

The promoter or promoter control element of the present invention may be operably linked to a polynucleotide to be transcribed. In this manner, the promoter or promoter control element may modify transcription by modulating transcript levels of that polynucleotide when inserted into a genome.

However, prior to insertion into a genome, the promoter or promoter control element need not be linked, operably or otherwise, to a polynucleotide to be transcribed. For example, the promoter or promoter control element may be inserted alone into the genome in front of a polynucleotide already present in the genome. In this manner, the promoter or promoter control element may modulate the transcription of a polynucleotide that was already present in the genome. This polynucleotide may be native to the genome or inserted at an earlier time.

Alternatively, the promoter or promoter control element may be inserted into a genome alone to modulate transcription. See, for example, Vaucheret, H et al. (1998) Plant J 16: 651-659. Rather, the promoter or promoter control element may be simply inserted into a genome or maintained extrachromosomally as a way to divert transcription resources of the system to itself. This approach may be used to downregulate the transcript levels of a group of polynucleotide(s).

The nature of the polynucleotide to be transcribed is not limited. Specifically, the polynucleotide may include sequences that will have activity as RNA as well as sequences that result in a polypeptide product. These sequences may include, but are not limited to antisense sequences, RNAi sequences, ribozyme sequences, spliceosomes, amino acid coding sequences, and fragments thereof. Specific coding sequences may include, but are not limited to endogenous proteins or fragments thereof, or heterologous proteins including marker genes or fragments thereof.

Constructs of the present invention would typically contain a promoter operably linked to a transcribable nucleic acid molecule operably linked to a 3′ transcription termination nucleic acid molecule. In addition, constructs may include but are not limited to additional regulatory nucleic acid molecules from the 3′-untranslated region (3′ UTR) of plant genes (e.g., a 3′ UTR to increase mRNA stability of the mRNA, such as the PI-II termination region of potato or the octopine or nopaline synthase 3′ termination regions). Constructs may include but are not limited to the 5′ untranslated regions (5′ UTR) of an mRNA nucleic acid molecule which can play an important role in translation initiation and can also be a genetic component in a plant expression construct. For example, non-translated 5′ leader nucleic acid molecules derived from heat shock protein genes have been demonstrated to enhance gene expression in plants (see for example, U.S. Pat. No. 5,659,122 and U.S. Pat. No. 5,362,865, all of which are hereby incorporated by reference). These additional upstream and downstream regulatory nucleic acid molecules may be derived from a source that is native or heterologous with respect to the other elements present on the promoter construct.

Thus, one embodiment of the invention is a promoter such as provided in SEQ ID NOs: 1-22, operably linked to a transcribable nucleic acid molecule so as to direct transcription of said transcribable nucleic acid molecule at a desired level or in a desired tissue or developmental pattern upon introduction of said construct into a plant cell. In some cases, the transcribable nucleic acid molecule comprises a protein-coding region of a gene, to and the promoter provides for transcription of a functional mRNA molecule that is translated and expressed as a protein product. Constructs may also be constructed for transcription of antisense RNA molecules or other similar inhibitory RNA in order to inhibit expression of a specific RNA molecule of interest in a target host cell.

Exemplary transcribable nucleic acid molecules for incorporation into constructs of the present invention include, for example, nucleic acid molecules or genes from a species other than the target gene species, or even genes that originate with or are present in the same species, but are incorporated into recipient cells by genetic engineering methods rather than classical reproduction or breeding techniques. Exogenous gene or genetic element is intended to refer to any gene or nucleic acid molecule that is introduced into a recipient cell. The type of nucleic acid molecule included in the exogenous nucleic acid molecule can include a nucleic acid molecule that is already present in the plant cell, a nucleic acid molecule from another plant, a nucleic acid molecule from a different organism, or a nucleic acid molecule generated externally, such as a nucleic acid molecule containing an antisense message of a gene, or a nucleic acid molecule encoding an artificial or modified version of a gene.

The promoters of the present invention can be incorporated into a construct using marker genes as described, and tested in transient analyses that provide an indication of gene expression in stable plant systems. As used herein the term “marker gene” refers to any transcribable nucleic acid molecule whose expression can be screened for or scored in some way. Methods of testing for marker gene expression in transient assays are known to those of skill in the art. Transient expression of marker genes has been reported using a variety of plants, tissues, and DNA delivery systems. For example, types of transient analyses can include but are not limited to direct gene delivery via electroporation or particle bombardment of tissues in any transient plant assay using any plant species of interest. Such transient systems would include but are not limited to electroporation of protoplasts from a variety of tissue sources or particle bombardment of specific tissues of interest. The present invention encompasses the use of any transient expression system to evaluate promoters or promoter fragments operably linked to any transcribable nucleic acid molecules, including but not limited to selected reporter genes, marker genes, or genes of agronomic interest. Examples of plant tissues envisioned to test in transients via an appropriate delivery system would include but are not limited to leaf base tissues, callus, cotyledons, roots, endosperm, embryos, floral tissue, pollen, and epidermal tissue.

Promoters and control elements of the present invention are useful for modulating metabolic or catabolic processes. Such processes include, but are not limited to, secondary product metabolism, amino acid synthesis, seed protein storage, oil development, pest defense and nitrogen usage. Some examples of genes, transcripts and peptides or polypeptides participating in these processes, which can be modulated by the present invention: are tryptophan decarboxylase (tdc) and strictosidine synthase (str1), dihydrodipicolinate synthase (DHDPS) and aspartate kinase (AK), 2S albumin and alpha-, beta-, and gamma-zeins, ricinoleate and 3-ketoacyl-ACP synthase (KAS), Bacillus thuringiensis (Bt) insecticidal protein, cowpea trypsin inhibitor (CpTI), asparagine synthetase and nitrite reductase. Alternatively, expression constructs can be used to inhibit expression of these peptides and polypeptides by incorporating the promoters in constructs for antisense use, co-suppression use or for the production of dominant negative mutations.

As explained above, several types of regulatory elements exist concerning transcription regulation. Each of these regulatory elements may be combined with the present vector if desired. Translation of eukaryotic mRNA is often initiated at the codon that encodes the first methionine. Thus, when constructing a recombinant polynucleotide according to the present invention for expressing a protein product, it is preferable to ensure that the linkage between the 3′ portion, preferably including the TATA box, of the promoter and the polynucleotide to be transcribed, or a functional derivative thereof, does not contain any intervening codons which are capable of encoding a methionine.

The vector of the present invention may contain additional components. For example, an origin of replication allows for replication of the vector in a host cell. Additionally, homologous sequences flanking a specific sequence allow for specific recombination of the specific sequence at a desired location in the target genome. T-DNA sequences also allow for insertion of a specific sequence randomly into a target genome.

The vector may also be provided with a plurality of restriction sites for insertion of a polynucleotide to be transcribed as well as the promoter and/or promoter control elements of the present invention. The vector may additionally contain selectable marker genes. The vector may also contain a transcriptional and translational initiation region, and a transcriptional and translational termination region functional in the host cell. The termination region may be native with the transcriptional initiation region, may be native with the polynucleotide to be transcribed, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; Joshi et al. (1987) Nucleic Acid Res. 15:9627-9639.

Where appropriate, the polynucleotide to be transcribed may be optimized for increased expression in a certain host cell. For example, the polynucleotide can be synthesized using preferred codons for improved transcription and translation. See U.S. Pat. Nos. 5,380,831, 5,436,391; see also and Murray et al. (1989) Nucleic Acids Res. 17:477-498.

Additional sequence modifications include elimination of sequences encoding spurious polyadenylation signals, exon intron splice site signals, transposon-like repeats, and other such sequences well characterized as deleterious to expression. The G-C content of the polynucleotide may be adjusted to levels average for a given cellular host, as calculated by reference to known genes expressed in the host cell. The polynucleotide sequence may be modified to avoid hairpin secondary mRNA structures.

A general description of expression vectors and reporter genes can be found in Gruber, et al. (1993) “Vectors for Plant Transformation” In Methods in Plant Molecular Biology & Biotechnology, Glich et al. Eds. pp. 89-119, CRC Press. Moreover GUS expression vectors and GUS gene cassettes are available from Clonetech Laboratories, Inc., Palo Alto, Calif. while luciferase expression vectors and luciferase gene cassettes are available from Promega Corp. (Madison, Wis.). GFP vectors are available from Aurora Biosciences.

### 6.3 Polynucleotide Insertion into a Host Cell

The promoters according to the present invention can be inserted into a host cell. A host cell includes but is not limited to a plant, mammalian, insect, yeast, and prokaryotic cell, preferably a plant cell.

The method of insertion into the host cell genome is chosen based on convenience. For example, the insertion into the host cell genome may either be accomplished by vectors that integrate into the host cell genome or by vectors which exist independent of the host cell genome.

The promoters of the present invention can exist autonomously or independent of the host cell genome. Vectors of these types are known in the art and include, for example, certain type of non-integrating viral vectors, autonomously replicating plasmids, artificial chromosomes, and the like.

Additionally, in some cases transient expression of a promoter may be desired.

The promoter sequences, promoter control elements or vectors of the present invention may be transformed into host cells. These transformations may be into protoplasts or intact tissues or isolated cells. Preferably expression vectors are introduced into intact tissue. General methods of culturing plant tissues are provided for example by Maki et al. (1993) “Procedures for Introducing Foreign DNA into Plants” In Methods in Plant Molecular Biology & Biotechnology, Glich et al. Eds. pp. 67-88 CRC Press; and by Phillips et al. (1988) “Cell-Tissue Culture and In-Vitro Manipulation” In Corn & Corn Improvement, 3rd Edition Sprague et al. eds., pp. 345-387, American Society of Agronomy Inc. et al.

Methods of introducing polynucleotides into plant tissue include the direct infection or co-cultivation of plant cell with Agrobacterium tumefaciens, Horsch et al. (1985) Science, 227:1229. Descriptions of Agrobacterium vector systems and methods for Agrobacterium-mediated gene transfer provided by Gruber et al. supra.

Alternatively, polynucleotides are introduced into plant cells or other plant tissues using a direct gene transfer method such as microprojectile-mediated delivery, DNA injection, electroporation and the like. More preferably polynucleotides are introduced into plant tissues using the microprojectile media delivery with the biolistic device. See, for example, Tomes et al., “Direct DNA transfer into intact plant cells via microprojectile bombardment” In: Gamborg and Phillips (Eds.) Plant Cell, Tissue and Organ Culture: Fundamental Methods, Springer Verlag, Berlin (1995).

Methods for specifically transforming dicots are well known to those skilled in the art. Transformation and plant regeneration using these methods have been described for a number of crops including, but not limited to, cotton (Gossypium hirsutum), soybean (Glycine max), peanut (Arachis hypogaea), and members of the genus Brassica.

Methods for transforming monocots are well known to those skilled in the art. Transformation and plant regeneration using these methods have been described for a number of crops including, but not limited to, barley (Hordeum vulgarae); maize (Zea mays); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), switchgrass (Panicum vigatum) and alfalfa (Medicago sativa). It is apparent to those of skill in the art that a number of transformation methodologies can be used and modified for production of stable transgenic plants from any number of target crops of interest.

In another embodiment of the current invention, expression constructs can be used for gene expression in callus culture for the purpose of expressing marker genes encoding peptides or polypeptides that allow identification of transformed plants. Here, a promoter that is operatively linked to a polynucleotide to be transcribed is transformed into plant cells and the transformed tissue is then placed on callus-inducing media. If the transformation is conducted with leaf discs, for example, callus will initiate along the cut edges. Once callus growth has initiated, callus cells can be transferred to callus shoot-inducing or callus root-inducing media. Gene expression will occur in the callus cells developing on the appropriate media: callus root-inducing promoters will be activated on callus root-inducing media, etc. Examples of such peptides or polypeptides useful as transformation markers include, but are not limited to barstar, glyphosate, chloramphenicol acetyltransferase (CAT), kanamycin, spectinomycin, streptomycin or other antibiotic resistance enzymes, green fluorescent protein (GFP), and β-glucuronidase (GUS), etc. Some of the promoters provided in SEQ ID NOs: 1-22 will also be capable of sustaining expression in some tissues or organs after the initiation or completion of regeneration. Examples of these tissues or organs are somatic embryos, cotyledon, hypocotyl, epicotyl, leaf, sterns, roots, flowers and seed.

Integration into the host cell genome also can be accomplished by methods to known in the art, for example, by the homologous sequences or T-DNA discussed above or using the cre-lox system (A. C. Vergunst et al. (1998) Plant Mol. Biol. 38:393).

### 7. USES OF THE PROMOTERS OF THE INVENTION

### 7.1 Use of the Promoters to Study and Screen for Expression

The promoters of the present invention can be used to further understand developmental mechanisms. For example, promoters that are specifically induced during callus formation, somatic embryo formation, shoot formation or root formation can be used to explore the effects of overexpression, repression or ectopic expression of target genes, or for isolation of trans-acting factors.

The vectors of the invention can be used not only for expression of coding regions but may also be used in exon-trap cloning, or promoter trap procedures to detect differential gene expression in various tissues (see Lindsey et al. (1993) Transgenic Research 2:3347. Auch and Reth (1990) Nucleic Acids Research 18: 6743).

Entrapment vectors, first described for use in bacteria (Casadaban and Cohen (1979) Proc. Nat. Aca. Sci. U.S.A. 76: 4530; Casadaban et al. (1980) J. Bacteriol. 143: 971) permit selection of insertional events that lie within coding sequences. Entrapment vectors can be introduced into pluripotent ES cells in culture and then passed into the germ line via chimeras (Gossler et al. aaa91989) Science 244: 463; Skarnes (1990) Biotechnology 8: 827). Promoter or gene trap vectors often contain a reporter gene, e.g., lacZ, lacking its own promoter and/or splice acceptor sequence upstream. That is, promoter gene traps contain a reporter gene with a splice site but no promoter. If the vector lands in a gene and is spliced into the gene product, then the reporter gene is expressed.

Recently, the isolation of preferentially-induced genes has been made possible with the use of sophisticated promoter traps (e.g. IVET) that are based on conditional auxotrophy complementation or drug resistance. In one IVET approach, various bacterial genome fragments are placed in front of a necessary metabolic gene coupled to a reporter gene. The DNA constructs are inserted into a bacterial strain otherwise lacking the metabolic gene, and the resulting bacteria are used to infect the host organism. Only bacteria expressing the metabolic gene survive in the host organism; consequently, inactive constructs can be eliminated by harvesting only bacteria that survive for some minimum period in the host. At the same time, broadly active constructs can be eliminated by screening only bacteria that do not express the reporter gene under laboratory conditions. The bacteria selected by such a method contain constructs that are selectively induced only during infection of the host. The IVET approach can be modified for use in plants to identify genes induced in either the bacteria or the plant cells upon pathogen infection or root colonization. For information on IVET see the articles by Mahan et al. (1993) Science 259:686-688, Mahan et al. (1995) Proc. Natl. Acad. Sci. USA 92:669-673, Heithoff et al. (1997) Proc. Natl. Acad. Sci USA 94:934-939, and Wang et al. (1996) Proc. Natl. Acad. Sci USA 93:10434.

### 7.2 Use of the Promoters to Transcribe Genes of Interest

In one embodiment of the invention, a nucleic acid molecule as shown in SEQ ID NOs: 1-22 is incorporated into a construct such that a promoter of the present invention is operably linked to a transcribable nucleic acid molecule that is a gene of agronomic interest. As used herein, the term “gene of agronomic interest” refers to a transcribable nucleic acid molecule that includes but is not limited to a gene that provides a desirable characteristic associated with plant morphology, physiology, growth and development, yield, nutritional enhancement, disease or pest resistance, or environmental or chemical tolerance. The expression of a gene of agronomic interest is desirable in order to confer an agronomically important trait. A gene of agronomic interest that provides a beneficial agronomic trait to crop plants may be, for example, including, but not limited to genetic elements comprising herbicide resistance, increased yield, insect control, fungal disease resistance, virus resistance, nematode resistance, bacterial disease resistance, starch production, modified oils production, high oil production, modified fatty acid content, high protein production, fruit ripening, enhanced animal and human nutrition, biopolymers, environmental stress resistance, pharmaceutical peptides, improved processing traits, improved digestibility, industrial enzyme production, improved flavor, nitrogen fixation, hybrid seed production, and biofuel production. The genetic elements, methods, and transgenes described in the patents listed above are hereby incorporated by reference.

Alternatively, a transcribable nucleic acid molecule can effect the above mentioned phenotypes by encoding a RNA molecule that causes the targeted inhibition of expression of an endogenous gene, for example via antisense, inhibitory RNA (RNAi), or cosuppression-mediated mechanisms. The RNA could also be a catalytic RNA molecule (i.e., a ribozyme) engineered to cleave a desired endogenous mRNA product. Thus, any nucleic acid molecule that encodes a protein or mRNA that expresses a phenotype or morphology change of interest may be useful for the practice of the present invention.

### 7.3. Stress Induced Preferential Transcription

Promoters and control elements providing modulation of transcription under oxidative, drought, oxygen, wound, and methyl jasmonate stress are particularly useful for producing host cells or organisms that are more resistant to biotic and abiotic stresses. In a plant, for example, modulation of genes, transcripts, and/or polypeptides in response to oxidative stress can protect cells against damage caused by oxidative agents, such as hydrogen peroxide and other free radicals.

Drought induction of genes, transcripts, and/or polypeptides are useful to increase the viability of a plant, for example, when water is a limiting factor. In contrast, genes, transcripts, and/or polypeptides induced during oxygen stress can help the flood tolerance of a plant.

The promoters and control elements of the present invention can modulate stresses similar to those described in, for example, stress conditions are VuPLD1 (drought stress; Cowpea; see Pham-Thi et al. (1999) Plant Mol Biol 39:1257-65), pyruvate decarboxylase (oxygen stress; rice; see Rivosal et al. (1997) Plant Physiol 114(3): 1021-29), chromoplast specific carotenoid gene (oxidative stress; capsicum; see Bouvier et al. (1998) J Biol Chem 273: 30651-59).

Promoters and control elements providing preferential transcription during wounding or induced by methyl jasmonate can produce a defense response in host cells or organisms. In a plant, for example, preferential modulation of genes, transcripts, and/or polypeptides under such conditions is useful to induce a defense response to mechanical wounding, pest or pathogen attack or treatment with certain chemicals.

Promoters and control elements of the present invention also can trigger a response similar to those described for cf9 (viral pathogen; tomato; see O'Donnell et al. (1998) Plant J 14(1): 137-42), hepatocyte growth factor activator inhibitor type 1 (HAI-1), which enhances tissue regeneration (tissue injury; human; Koono et al. (1999) J Histochem Cytochem 47: 673-82), copper amine oxidase (CuAO), induced during ontogenesis and wound healing (wounding; chick-pea; Rea et al. (1998) FEBS Lett 437: 177-82), proteinase inhibitor II (wounding; potato; see Pena-Cortes et al. (1988) Planta 174: 84-89), protease inhibitor II (methyl jasmonate; tomato; see Farmer and Ryan (1990) Proc Natl Acad Sci USA 87: 7713-7716), two vegetative storage protein genes VspA and VspB (wounding, jasmonic acid, and water deficit; soybean; see Mason and Mullet (1990) Plant Cell 2: 569-579).

Up-regulation and transcription down-regulation are useful for these applications. For instance, genes, transcripts, and/or polypeptides that increase oxidative, flood, or drought tolerance may require up-regulation of transcription.

Typically, promoter or control elements, which provide preferential transcription in wounding or under methyl jasmonate induction, produce transcript levels that are statistically significant as compared to cell types, organs or tissues under other conditions.

For preferential up-regulation of transcription, promoter and control elements produce transcript levels that are above background of the assay.

### 7.4. Light Induced Preferential Transcription

Promoters and control elements providing preferential transcription when induced by light exposure can be utilized to modulate growth, metabolism, and development; to increase drought tolerance; and decrease damage from light stress for host cells or organisms. In a plant, for example, modulation of genes, transcripts, and/or polypeptides in response to light is useful
(1) to increase the photosynthetic rate;
(2) to increase storage of certain molecules in leaves or green parts only, e.g. silage with high protein or starch content;
(3) to modulate production of exogenous compositions in green tissue, e.g. certain feed enzymes;
(4) to induce growth or development, such as fruit development and maturity, during extended exposure to light;
(5) to modulate guard cells to control the size of stomata in leaves to prevent water loss, or
(6) to induce accumulation of beta-carotene to help plants cope with light induced stress.

The promoters and control elements of the present invention also can trigger responses similar to those described in: abscisic acid insensitive3 (ABI3) (dark-grown Arabidopsis seedlings, see Rohde et al. (2000) Plant Cell 12: 35-52), asparagine synthetase (pea root nodules, see Tsai and Coruzzi (1990) EMBO J 9: 323-32), mdm2 gene (human tumor, see Saucedo et al. (1998) Cell Growth Differ 9: 119-30).

Up-regulation and transcription down-regulation are useful for these applications. For instance, genes, transcripts, and/or polypeptides that increase drought or light tolerance may require up-regulation of transcription.

Typically, promoter or control elements, which provide preferential transcription in cells, tissues or organs exposed to light, produce transcript levels that are statistically significant as compared to cells, tissues, or organs under decreased light exposure (intensity or length of time).

For preferential up-regulation of transcription, promoter and control elements produce transcript levels that are above background of the assay.

### 7.5. Dark Induced Preferential Transcription

Promoters and control elements providing preferential transcription when induced by dark or decreased light intensity or decreased light exposure time can be utilized to time growth, metabolism, and development, to modulate photosynthesis capabilities for host cells or organisms. In a plant, for example, modulation of genes, transcripts, and/or polypeptides in response to dark is useful, for example,
(1) to induce growth or development, such as fruit development and maturity, despite lack of light;
(2) to modulate genes, transcripts, and/or polypeptide active at night or on cloudy days; or
(3) to preserve the plastid ultra structure present at the onset of darkness.

The present promoters and control elements can also trigger response similar to those described in the section above.

Up-regulation and transcription down-regulation is useful for these applications. For instance, genes, transcripts, and/or polypeptides that increase or decrease growth and development may require up-regulation of transcription.

Typically, promoter or control elements, which provide preferential transcription under exposure to dark or decrease light intensity or decrease exposure time, produce transcript levels that are statistically significant.

For preferential up-regulation of transcription, promoter and control elements produce transcript levels that are above background of the assay.

### 7.6. Leaf Preferential Transcription

Promoters and control elements providing preferential transcription in a leaf can modulate growth, metabolism, and development or modulate energy and nutrient utilization in host cells or organisms. In a plant, for example, preferential modulation of genes, transcripts, and/or polypeptide in a leaf, is useful, for example,
(1) to modulate leaf size, shape, and development;
(2) to modulate the number of leaves; or
(3) to modulate energy or nutrient usage in relation to other organs and tissues

Up-regulation and transcription down-regulation is useful for these applications. For instance, genes, transcripts, and/or polypeptides that increase growth, for example, may require up-regulation of transcription.

Typically, promoter or control elements, which provide preferential transcription in the cells, tissues, or organs of a leaf, produce transcript levels that are statistically significant as compared to other cells, organs or tissues.

For preferential up-regulation of transcription, promoter and control elements produce transcript levels that are above background of the assay.

### 7.7. Root Preferential Transcription

Promoters and control elements providing preferential transcription in a root can modulate growth, metabolism, development, nutrient uptake, nitrogen fixation, or modulate energy and nutrient utilization in host cells or organisms. In a plant, for example, preferential modulation of genes, transcripts, and/or in a leaf, is useful
(1) to modulate root size, shape, and development;
(2) to modulate the number of roots, or root hairs;
(3) to modulate mineral, fertilizer, or water uptake;
(4) to modulate transport of nutrients; or
(4) to modulate energy or nutrient usage in relation to other organs and tissues.

Up-regulation and transcription down-regulation is useful for these applications. For instance, genes, transcripts, and/or polypeptides that increase or decrease growth, for example, may require up-regulation of transcription.

Typically, promoter or control elements, which provide preferential transcription in cells, tissues, or organs of a root, produce transcript levels that are statistically significant as compared to other cells, organs or tissues.

For preferential up-regulation of transcription, promoter and control elements produce transcript levels that are above background of the assay.

### 7.8. Stem/Shoot Preferential Transcription

Promoters and control elements providing preferential transcription in a stem or shoot can modulate growth, metabolism, and development or modulate energy and nutrient utilization in host cells or organisms. In a plant, for example, preferential modulation of genes, transcripts, and/or polypeptide in a stem or shoot, is useful, for example,
(1) to modulate stem/shoot size, shape, and development; or
(2) to modulate energy or nutrient usage in relation to other organs and tissues

Up-regulation and transcription down-regulation is useful for these applications. For instance, genes, transcripts, and/or polypeptides that increase growth, for example, may require up-regulation of transcription.

Typically, promoter or control elements, which provide preferential transcription in the cells, tissues, or organs of a stem or shoot, produce transcript levels that are statistically significant as compared to other cells, organs or tissues.

For preferential up-regulation of transcription, promoter and control elements produce transcript levels that are above background of the assay.

### 7.9. Fruit and Seed Preferential Transcription

Promoters and control elements providing preferential transcription in a silique or fruit can time growth, development, or maturity; or modulate fertility; or modulate energy and nutrient utilization in host cells or organisms. In a plant, for example, preferential modulation of genes, transcripts, and/or polypeptides in a fruit, is useful
(1) to modulate fruit size, shape, development, and maturity;
(2) to modulate the number of fruit or seeds;
(3) to modulate seed shattering;
(4) to modulate components of seeds, such as, storage molecules, starch, protein, oil, vitamins, anti-nutritional components, such as phytic acid;
(5) to modulate seed and/or seedling vigor or viability;
(6) to incorporate exogenous compositions into a seed, such as lysine rich proteins;
(7) to permit similar fruit maturity timing for early and late blooming flowers; or
(8) to modulate energy or nutrient usage in relation to other organs and tissues.

Up-regulation and transcription down-regulation is useful for these applications. For instance, genes, transcripts, and/or polypeptides that increase or decrease growth, for example, may require up-regulation of transcription.

Typically, promoter or control elements, which provide preferential transcription in the cells, tissues, or organs of siliques or fruits, produce transcript levels that are statistically significant as compared to other cells, organs or tissues.

For preferential up-regulation of transcription, promoter and control elements produce transcript levels that are above background of the assay.

### 7.10. Callus Preferential Transcription

Promoters and control elements providing preferential transcription in a callus can be useful to modulating transcription in dedifferentiated host cells. In a plant transformation, for example, preferential modulation of genes, transcripts, in callus is useful to modulate transcription of a marker gene, which can facilitate selection of cells that are transformed with exogenous polynucleotides.

Up-regulation and transcription down-regulation is useful for these applications. For instance, genes, transcripts, and/or polypeptides that increase marker gene detectability, for example, may require up-regulation of transcription.

For preferential up-regulation of transcription, promoter and control elements produce transcript levels that are above background of the assay.

### 7.11. Flower Specific Transcription

Promoters and control elements providing preferential transcription in flowers can modulate pigmentation; or modulate fertility in host cells or organisms. In a plant, for example, preferential modulation of genes, transcripts, and/or polypeptides in a flower, is useful,
(1) to modulate petal color; or
(2) to modulate the fertility of pistil and/or stamen.

Up-regulation and transcription down-regulation is useful for these applications. For instance, genes, transcripts, and/or polypeptides that increase or decrease pigmentation, for example, may require up-regulation of transcription

Typically, promoter or control elements, which provide preferential transcription in flowers, produce transcript levels that are statistically significant as compared to other cells, organs or tissues.

For preferential up-regulation of transcription, promoter and control elements produce transcript levels that are above background of the assay.

### 7.12. Immature Bud and Inflorescence Preferential Transcription

Promoters and control elements providing preferential transcription in a immature bud or inflorescence can time growth, development, or maturity; or modulate fertility or viability in host cells or organisms. In a plant, for example, preferential modulation of genes, transcripts, and/or polypeptide in a fruit, is useful,
(1) to modulate embryo development, size, and maturity;
(2) to modulate endosperm development, size, and composition;
(3) to modulate the number of seeds and fruits; or
(4) to modulate seed development and viability.

Up-regulation and transcription down-regulation is useful for these applications. For instance, genes, transcripts, and/or polypeptides that increase or decrease growth, for example, may require up-regulation of transcription.

Typically, promoter or control elements, which provide preferential transcription in immature buds and inflorescences, produce transcript levels that are statistically significant as compared to other cell types, organs or tissues.

For preferential up-regulation of transcription, promoter and control elements produce transcript levels that are above background of the assay.

### 7.13. Senescence Preferential Transcription

Promoters and control elements providing preferential transcription during senescence can be used to modulate cell degeneration, nutrient mobilization, and scavenging of free radicals in host cells or organisms. Other types of responses that can be modulated include, for example, senescence associated genes (SAG) that encode enzymes thought to be involved in cell degeneration and nutrient mobilization (Arabidopsis; see Hensel et al. (1993) Plant Cell 5: 553-64), and the CP-2/cathepsin L gene (rat; Kim and Wright (1997) Biol Reprod 57: 1467-77), both induced during senescence.

In a plant, for example, preferential modulation of genes, transcripts, and/or to polypeptides during senescencing is useful to modulate fruit ripening.

Up-regulation and transcription down-regulation is useful for these applications. For instance, genes, transcripts, and/or polypeptides that increase or decrease scavenging of free radicals, for example, may require up-regulation of transcription.

Typically, promoter or control elements, which provide preferential transcription in cells, tissues, or organs during senescence, produce transcript levels that are statistically significant as compared to other conditions.

For preferential up-regulation of transcription, promoter and control elements produce transcript levels that are above background of the assay.

### 7.14. Germination Preferential Transcription

Promoters and control elements providing preferential transcription in a germinating seed can time growth, development, or maturity; or modulate viability in host cells or organisms. In a plant, for example, preferential modulation of genes, transcripts, and/or polypeptide in a germinating seed, is useful,
(1) to modulate the emergence of they hypocotyls, cotyledons and radical; or
(2) to modulate shoot and primary root growth and development;

Up-regulation and transcription down-regulation is useful for these applications. For instance, genes, transcripts, and/or polypeptides that increase or decrease growth, for example, may require up-regulation of transcription.

Typically, promoter or control elements, which provide preferential transcription in a germinating seed, produce transcript levels that are statistically significant as compared to other cell types, organs or tissues.

For preferential up-regulation of transcription, promoter and control elements produce transcript levels that are above background of the assay.

### 8. GFP EXPERIMENTAL PROCEDURES AND RESULTS PROCEDURES

The polynucleotide sequences of the present invention were tested for promoter activity using Green Fluorescent Protein (GFP) assays in the following manner.

Approximately 1-3 kb of genomic sequence occurring immediately upstream of the ATG translational start site of the gene of interest was isolated using appropriate primers tailed with BstXI restriction sites. Standard PCR reactions using these primers and genomic DNA were conducted. The resulting product was isolated, cleaved with BstXI and cloned into the BstXI site of an appropriate vector, such as pNewBin4-HAP1-GFP (see FIG. 1).

### Agrobacterium-Mediated Transformation of Arabidopsis

Host Plants and Transgenes: Wild-type Arabidopsis thaliana Wassilewskija (WS) plants are transformed with Ti plasmids containing nucleic acid sequences to be expressed, as noted in the respective examples, in the sense orientation relative to the 35S promoter in a Ti plasmid. A Ti plasmid vector useful for these constructs, CRS 338, contains the Ceres-constructed, plant selectable marker gene phosphinothricin acetyltransferase (PAT), which confers herbicide resistance to transformed plants.

Ten independently transformed events are typically selected and evaluated for their qualitative phenotype in the T₁ generation.

Preparation of Soil Mixture: 24 L Sunshine Mix #5 soil (Sun Gro Horticulture, Ltd., Bellevue, Wash.) is mixed with 16 L Therm-O-Rock vermiculite (Therm-O-Rock West, Inc., Chandler, Ariz.) in a cement mixer to make a 60:40 soil mixture. To the soil mixture is added 2 Tbsp Marathon 1% granules (Hummert, Earth City, Mo.), 3 Tbsp OSMOCOTE® 14-14-14 (Hummert, Earth City, Mo.) and 1 Tbsp Peters fertilizer 20-20-20 (J.R. Peters, Inc., Allentown, Pa.), which are first added to 3 gallons of water and then added to the soil and mixed thoroughly. Generally, 4-inch diameter pots are filled with soil mixture. Pots are then covered with 8-inch squares of nylon netting.

Planting: Using a 60 mL syringe, 35 mL of the seed mixture is aspirated. 25 drops are added to each pot. Clear propagation domes are placed on top of the pots that are then placed under 55% shade cloth and subirrigated by adding 1 inch of water.

Plant Maintenance: 3 to 4 days after planting, lids and shade cloth are removed. Plants are watered as needed. After 7-10 days, pots are thinned to 20 plants per pot using forceps. After 2 weeks, all plants are subirrigated with Peters fertilizer at a rate of 1 Tsp per gallon of water. When bolts are about 5-10 cm long, they are clipped between the first node and the base of stem to induce secondary bolts. Dipping infiltration is performed 6 to 7 days after clipping.

Preparation of Agrobacterium: To 150 mL fresh YEB is added 0.1 mL each of carbenicillin, spectinomycin and rifampicin (each at 100 mg/ml stock concentration). Agrobacterium starter blocks are obtained (96-well block with Agrobacterium cultures grown to an OD₆₀₀ of approximately 1.0) and inoculated one culture vessel per construct by transferring 1 mL from appropriate well in the starter block. Cultures are then incubated with shaking at 27° C. Cultures are spun down after attaining an OD₆₀₀ of approximately 1.0 (about 24 hours). 200 mL infiltration media is added to resuspend Agrobacterium pellets. Infiltration media is prepared by adding 2.2 g MS salts, 50 g sucrose, and 5 μL 2 mg/ml benzylaminopurine to 900 ml water.

Dipping Infiltration: The pots are inverted and submerged for 5 minutes so that the aerial portion of the plant is in the Agrobacterium suspension. Plants are allowed to grow normally and seed is collected.

High-throughput Screening of T₁ Transgenic Plants: Seed is evenly dispersed into water-saturated soil in pots and placed into a dark 4° C. cooler for two nights to promote uniform germination. Pots are then removed from the cooler and covered with 55% shade cloth for 4-5 days. Cotyledons are fully expanded at this stage. FINALE® (Sanofi Aventis, Paris, France) is sprayed on plants (3 ml FINALE® diluted into 48 oz. water) and repeated every 3-4 days until only transformants remain.

### GFP Assay

Tissues are dissected by eye or under magnification using INOX 5 grade forceps and placed on a slide with water and coversliped. An attempt is made to record images of observed expression patterns at earliest and latest stages of development of tissues listed below. Specific tissues will be preceded with High (H), Medium (M), Low (L) designations.

| | |
|---|---|
| Flower | Pedicel, receptacle, nectary, sepal, petal, filament,t anther, pollen, carpel, style, |
| | papillae, vascular, epidermis, stomata, trichome |
| Silique | Stigma, style, carpel, septum, placentae, transmitting tissue, vascular, epidermis, |
| | stomata, abscission zone, ovule |
| Ovule | Pre-fertilization: inner integument,t outer integument, embryo sac, funiculus, |
| | chalaza, micropyle, gametophyte |
| | Post-fertilization: zygote, inner integument, outer integument, seed coat, |
| | primordia, chalaza, micropyle, early endosperm, mature endosperm, embryo |
| Embryo | Suspensor, preglobular, globular, heart, torpedo, late mature, provascular, |
| | hypophysis, radicle, cotyledons, hypocotyl |
| Stem | Epidermis, cortex, vascular, xylem, phloem, pith, stomata, trichome |
| Leaf | Petiole, mesophyl,l vascular, epidermis, trichome, primordia, stomata, stipul,e |
| | margin |

T1 Mature: These are the T1 plants resulting from independent transformation events. These are screened between stage 6.50-6.90 (i.e. the plant is flowering and 50-90% of the flowers that the plant will make have developed), which is 4-6 weeks of age. At this stage the mature plant possesses flowers, siliques at all stages of development, and fully expanded leaves. The plants are initially imaged under UV with a Leica Confocal microscope to allow examination of the plants on a global level. If expression is present, they are re-imaged using scanning laser confocal micsrocopy.

T2 Seedling: Progeny are collected from the T1 plants giving the same expression pattern and the progeny (T2) are sterilized and plated on agar-solidified medium containing M&S salts. In the event that there is no expression in the T1 plants, T2 seeds are planted from all lines. The seedlings are grown in Percival incubators under continuous light at 22° C. for 10-12 days. Cotyledons, roots, hypocotyls, petioles, leaves, and the shoot meristem region of individual seedlings were screened until two seedlings were observed to have the same pattern. In general, the same expression pattern was found in the first two seedlings. However, up to 6 seedlings were screened before “no expression pattern” was recorded. All constructs are screened as T2 seedlings even if they did not have an expression pattern in the T1 generation.

T2 Mature: The T2 mature plants were screened in a similar manner to the T1 plants. The T2 seeds were planted in the greenhouse, exposed to selection and at least one plant screened to confirm the T1 expression pattern. In instances where there were any subtle changes in expression, multiple plants were examined and the changes noted in the tables.

T3 Seedling: This was done similar to the T2 seedlings except that only the plants for which we are trying to confirm the pattern are planted.

### Image Data:

Images are collected by scanning laser confocal microscopy. Scanned images are taken as 2-D optical sections or 3-D images generated by stacking the 2-D optical sections collected in series. All scanned images are saved as TIFF files by imaging software, edited in Adobe Photoshop, and labeled in Powerpoint specifying organ and specific expressing tissues.

### Results

The Promoter Expression Reports of Table 1 present the results of the GFP assays as reported by their corresponding construct number and line number.

Promoter Expression Report #324.PT1026.CC

Promoter Tested In: Arabidopsis thaliana, Wassilewskija (WS) ecotype
Spatial expression summary:

| | |
|---|---|
| Flower | H pedicel H receptacle H nectary H sepal H petal H filament L anther H |
| | carpel |
| | H style H papillae H vascular H epidermis H stomata L trichome H silique |
| Silique | H stigma H style H carpel H septum H placentae H funiculus H vascular |
| | H epidermis H stomata H abscission zone H ovule |
| Ovule | Pre-fertilization: H outer integument H funiculus H chalaza |
| | Post-fertilization: H funiculus H inner integument H outer integument |
| | H seed coat H chalaza |
| Embryo | H heart H torpedo H late H mature H radicle H cotyledons |
| Stem | H epidermis H cortex H interfascicular region H vascular H xylem |
| | H phloem |
| | H pith H stomata |
| Leaf | H petiole H mesophyll H vascular H epidermis L trichome H stomata |
| Shoot apical | H Shoot apical meristem H Flower primordium |
| meristem | |
| Hypocotyl | H epidermis H cortex H vascular H xylem H phloem H stomata |
| Cotyledon | H mesophyll H vascular H epidermis |
| Rosette Leaf | H mesophyll H vascular H epidermis H petiole H primordia |
| Primary Root | H epidermis H cortex H endodermis H vascular H xylem H phloem |
| | H pericycle H quiescent L root hairs |

Observed expression pattern:
T1 Mature expression: Broadly expressed GFP expression. High GFP expression throughout
mature tissues. High GFP expression at the inflorescence meristem and flowers. High GFP
expression in epidermis, cortex, vascular, vascular bundles and parenchyma cells of stem. High
GFP expression in epidermis, mesophyll and vasculature of leaf. High GFP expression in anther
wall. Not expressed in pollen. High GFP expression in heart to mature stage embryos.
T2 Seedling expression: High GFP expression in epidermis, mesophyll and vasculature of
cotyledons and rosette leaves. High GFP expression in root at transition zone decreasing toward
root tip. GFP expressed in meristem cells at root tip. Low GFP expression in root hairs.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 8 Events Expressing: n = 5

| | |
|---|---|
| X Flower | H pedicel H receptacle H nectary H sepal H petal H filament L anther |
| | □tapetum □pollen H carpel H style H papillae H vascular H epidermis |
| | H stomata L trichome |
| | H silique |
| X Silique | H stigma H style H carpel H septum H placentae H funiculus |
| | □transmitting tissue H vascular H epidermis H stomata H abscission |
| | zone H ovule |
| X Ovule | Pre-fertilization: □primordia □inner integument H outer integument |
| | □embryo sac H funiculus H chalaza □micropyle □gametophyte |
| | Post-fertilization: □zygote □suspensor □ embryo sack H funiculus H |
| | inner integument H outer integument □ endothelium H seed coat |
| | □primordia H chalaza □micropyle □early endosperm □mature |
| | endosperm □embryo |
| X Embryo | □suspensor □preglobular □globular H heart H torpedo H late H mature |
| | □provascular □hypophysis H radicle H cotyledons □root meristem |
| | □shoot meristem |
| X Stem | H epidermis H cortex H interfascicular region H vascular H xylem H |
| | phloem H pith H stomata □trichome |
| X Leaf | H petiole H mesophyll H vascular H epidermis L trichome □primordia |
| | H stomata □stipule □margin |
| X Shoot apical | H Shoot apical meristem H Flower primordium |
| meristem | |

Table 2. T2 Seedling Expression Tissues Screened
Events Screened: n = 3 Events Expressing: n = 1

| | |
|---|---|
| X Hypocotyl | H epidermis H cortex H vascular H xylem H phloem H stomata |
| X Cotyledon | H mesophyll H vascular H epidermis □margin □petiole □ stomata |
| | □hydathode |
| X Rosette Leaf | H mesophyll H vascular H epidermis □trichome H petiole |
| | H primordia □stomata □stipule □margin □hydathode |
| X Primary Root | H epidermis □trichoblast □atrichoblast H cortex H endodermis |
| | H vascular H xylem H phloem H pericycle H quiescent |
| | □columella □ root cap L root hairs |

Table 3. T2 Mature Plant Expression Organs/Tissues screened
Events Screened: n = 3 Events Expressing: n = 1
Guard cell expression observed

| | |
|---|---|
| Table 4. | Promoter utility |
| Utility: | Among other uses this promoter sequence could be useful to |
| | improve: Herbicide resistance, antibiotic resistance, insect resistance, |
| | virus resistance, fungal resistance, nematode resistance, |
| | abiotic stress resistance, nutrient utilization, delayed |
| | senescence, protein synthesis, chemical synthesis, modulating |
| | gene expression, antibiotic resistance gene expression, |
| | herbicide resistance gene expression, transformation efficiency, |
| | plant biomass, plant architecture, organ number, organ size, |
| | photosynthesis, source strength, seed number, seed size, seed |
| | yield, modulate flowering time, modulate flower number. |

2. Jagdeep S. Sandhu, Carl I. Webster, John C. Gray, A/T-rich sequences act as quantitative
enhancers of gene expression in transgenic tobacco and potato plants, Plant Molecular Biology,
Volume 37, Issue 5, July 1998, Pages 885-896

| | |
|---|---|
| Construct: | PT1026 |
| Promoter candidate I.D: | 22205547 |
| Events expressing: | −02, −04, −05, −07, −08 |

Promoter Expression Report #137.PT0513

Promoter Tested In: Arabidopsis thaliana, Wassilewskija (WS) ecotype
Spatial expression summary:

| | |
|---|---|
| Flower | H stomata |
| Silique | H stomata |
| Stem | H stomata |
| Leaf | H stomata |
| Hypocotyl | L vascular |
| Primary Root | L epidermis L vascular |
| Lateral root | L initials |

Observed expression pattern:
T1 mature: High guard cell expression throughout all organs.
T2 seedling: Low GFP expression in root epidermis, vascular and lateral root initial cells.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Inductions completed.

| | | | | | | |
|---|---|---|---|---|---|---|
| Treatment: | Date: | Age: | Gen: | Time points: | Events | Response: |
| | | | | | Screened/ | |
| | | | | | Response | |
| 1. Drought | | 4 wks | T2 | 8 days no water | 2/0 | No |

Table 1. T1 Mature Plant Expression Organs/Tissues screened
Events Screened: n = 2 Events Expressing: n = 2

| | |
|---|---|
| X Flower | □pedicel □receptacle □nectary □sepal □petal □filament □anther |
| | □pollen □carpel □style □papillae □vascular □epidermis H stomata |
| | □trichome □silique |
| X Silique | □stigma □style □carpel □septum □placentae □transmitting tissue |
| | □vascular □epidermis H stomata □abscission zone □ovule |
| X Stem | □epidermis □cortex □vascular □xylem □phloem □pith H stomata |
| | □trichome |
| X Leaf | □petiole □mesophyll □vascular □epidermis □trichome □primordia |
| | H stomata □stipule □margin |

Table 2. T2 Seedling Expression Tissues Screened
Events Screened: n = 3 Events Expressing: n = 2

| | |
|---|---|
| X Hypocotyl | □epidermis □cortex L vascular □xylem □phloem □stomata |
| X Primary Root | L epidermis □trichoblast □atrichoblast □cortex □endodermis |
| | Lvascular □xylem □phloem □ pericycle □quiescent |
| | □columella □root cap □root hairs |
| X Lateral root | □epidermis □trichoblast □atrichoblast □cortex □endodermis |
| | L initials □flanking cells □vascular □lateral root cap |

Table 3. T2 Mature Plant Expression Organs/Tissues screened
Events Screened: n = 3 Events Expressing: n = 3

| | |
|---|---|
| Table 4. | Promoter utility |
| Utility: | Drought tolerance, nitrogen use efficiency, nitrogen uptake, and |
| | seedling establishment |
| Construct: | PT0513 |
| Promoter candidate I.D: | 11768790 |
| Lines expressing: | −01, −02, −03 |

Promoter Expression Report #152.PT590.CC

Promoter Tested In: Arabidopsis thaliana, Wassilewskija (WS) ecotype
Spatial expression summary:

| | |
|---|---|
| Flower | H stomata |
| Silique | H stomata |
| Rosette Leaf | H stomata H stipule |
| Primary Root | L epidermis L trichoblast H cortex L root hairs |

Observed expression pattern:
T1 mature: Guard cell expression throughout inflorescence apex and carpels in early flower
buds.
T2 seedling: GFP expression specific within cortex cells overlaying lateral root primordia and
root hair producing epidermal cells.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling DT2 Mature □T3 Seedling |

Inductions completed.

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | 8 days no water | 2/0 | No |
| 2. Far red | 7 days | T2 | 1 Hr | 2/0 | No |
| Far Red₇₃₀ = 525 | | | 4 Hr | 2/0 | No |
| □W/cm² | | | 24 Hr | 2/0 | No |

Inducible expression summary:

| | | | |
|---|---|---|---|
| Treatment: | Time point induced: | Organs induced: | Tissues induced: |

Table 1. T1 Mature Plant Expression Organs/Tissues screened
Events Screened: n = 6 Events Expressing: n = 2

| | |
|---|---|
| X Flower | □pedicel □receptacle □nectary □sepal □petal □filament □anther |
| | □pollen □carpel □style □papillae □vascular □epidermis H stomata |
| | □trichome □silique |
| X Silique | □stigma □style □carpel □septum □placentae □transmitting tissue |
| | □vascular □epidermis H stomata □abscission zone □ovule |

Table 2. T2 Seedling Expression Tissues Screened
Events Screened: n = 2 Events Expressing: n = 2
Seedlings expressing/Seedlings screened
Event-01: 5/6
Event-02: 4/6

| X Rosette Leaf | □mesophyll □vascular □epidermis □trichome □petiole |
|---|---|
| | □primordia H stomata H stipule □margin □hydathode |
| X Primary Root | L epidermis L trichoblast □atrichoblast H cortex □endodermis |
| | □vascular □xylem □phloem □pericycle □quiescent |
| | □columella □root cap L root hairs |
| Table 3. | Promoter utility |
| Utility: | Among other uses this promoter sequence could be useful to |
| | improve: Drought tolerance, nitrogen use efficiency, nitrogen |
| | uptake, and seedling establishment |
| Construct: | PT0590 |
| Promoter candidate I.D: | 11768848 |
| Lines expressing: | PT0590 −03, −04 |

Promoter Expression Report #191.PT0850.CC

Promoter Tested In: Arabidopsis thaliana, Wassilewskija (WS) ecotype
Spatial expression summary:

| | |
|---|---|
| Flower | M stomata |
| Stem | M stomata |
| Hypocotyl | M stomata |
| Cotyledon | M stomata |
| Rosette Leaf | M stomata |

Observed expression pattern:
T1 mature: Guard cell expression throughout stem and pedicels.
T2 seedling: Guard cell expression throughout seedling.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling □T2 Mature □T3 Seedling |

Table 1. T1 Mature Plant Expression Organs/Tissues screened
Events Screened: n = 3 Events Expressing: n = 2

| | |
|---|---|
| X Flower | □pedicel □receptacle □nectary □sepal □petal □filament □anther |
| | □pollen □carpel □style □papillae □vascular □epidermis M stomata |
| | □trichome □silique |
| X Stem | □epidermis □cortex □vascular □xylem □phloem □pith M stomata |
| | □trichome |

Table 2. T2 Seedling Expression Tissues Screened
Events Screened: n = 3 Events Expressing: n = 2
Seedlings expressing/Seedlings screened
Event-01: 0/6
Event-02: 4/6
Event-04: 5/6

| X Hypocotyl | □epidermis □cortex □vascular □xylem □phloem M stomata |
|---|---|
| X Cotyledon | □mesophyll □vascular □epidermis □margin M stomata |
| | □hydathode |
| X Rosette Leaf | □mesophyll □vascular □epidermis □trichome □petiole |
| | □primordia M stomata □stipule □margin □hydathode |
| Table 3. | Promoter utility |
| Utility: | Among other uses this promoter sequence could be useful to |
| | improve: Drought tolerance, nitrogen use efficiency, nitrogen |
| | uptake, and seedling establishment |
| Trait Area: | Water use efficiency, nutrients and light quality |
| Construct: | PT0850 |
| Promoter candidate I.D: | 15224215 |
| Lines expressing: | PT0850 −02, −04 |

Promoter Expression Report #212.PT0723

Promoter Tested In: Arabidopsis thaliana, Wassilewskija (WS) ecotype
Spatial expression summary:

| | |
|---|---|
| Ovule | Post-fertilization: H suspensor H embryo |
| Embryo | H suspensor H heart H torpedo H late H mature H hypophysis H radicle |
| | H cotyledons |
| Hypocotyl | L epidermis |

Observed expression pattern:
T1 mature: GFP expression specific to embryo. Highest expression at root cap in heart stage
through mature embryo.
T2 seedling: Low GFP expression in epidermis of hypocotyl.
T2 mature: GFP expression in embryo confirmed.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Inductions completed.

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | 8 days no water | 2/0 | No |
| 2. Drought | 4 wks | T2 | ~1.0% moisture | 6/0 | No |

Table 1. T1 Mature Plant Expression Organs/Tissues screened
Events Screened: n = 4 Events Expressing: n = 2

| | |
|---|---|
| X Ovule | Pre-fertilization: □primordia □inner integument □outer integument |
| | □embryo sac □funiculus □chalaza □micropyle □gametophyte |
| | Post-fertilization: □zygote H suspensor □ embryo sack □funiculus |
| | □inner integument □outer integument □endothelium □seed coat |
| | □primordia □chalaza □micropyle □early endosperm □mature |
| | endosperm H embryo |
| X Embryo | H suspensor □preglobular □globular H heart H torpedo H late H mature |
| | □provascular H hypophysis H radicle H cotyledons □hypocotyl |

Table 2. T2 Seedling Expression Tissues Screened
Events Screened: n = 6 Events Expressing: n = 3

| | |
|---|---|
| X Hypocotyl | L epidermis □cortex □vascular □xylem □phloem □stomata |

Table 3. T2 Mature Plant Expression Organs/Tissues screened
Events Screened: n = 2 Events Expressing: n = 2

| | |
|---|---|
| X Embryo | □suspensor □preglobular □globular □heart □torpedo □late H mature |
| | □provascular □hypophysis □radicle □cotyledons □root meristem |
| | □shoot meristem |
| X Aerial organs | □ inflorescence meristem □ shoot apical meristem □ flower primordium |
| | □silique □ovule H embryo □ stem □ leaf |

Table 3. Induction Screens
Drought: No drought expression was observed

| | |
|---|---|
| Table 4. | Promoter utility |
| Utility: | Among other uses this promoter sequence could be useful to |
| | improve: timing of seed germination, efficiency of |
| | germination, faster root growth and seedling establishment, |
| | seed tolerance to cold, and seed tolerance to desiccation and |
| | drought, seed composition. |
| Construct: | PT0723 |
| Promoter candidate I.D: | 15371692 |
| Lines expressing: | 01, 02, 03, 05 |

Promoter Expression Report#258.PT0769

Promoter Tested In: Arabidopsis thaliana, Wassilewskija (WS) ecotype
Spatial expression summary:

| | |
|---|---|
| Primary Root | L cortex |
| Mature root | L mature root |

Observed expression pattern:
T1 Mature expression: None observed.
T2 Seedling expression: Low GFP expressed in cortex cells of seedling root.
T2 Mature expression: Low GFP expression in root detected.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 3 Events Expressing: n = 0
X No GFP Expression Detected
Table 2. T2 Seedling Expression Tissues Screened
Events Screened: n = 6 Events Expressing: n = 2

| | |
|---|---|
| X Primary Root | □ epidermis □trichoblast □atrichoblast L cortex □ endodermis |
| | □ vascular □xylem □phloem □pericycle □quiescent |
| | □columella □ root cap □ root hairs |

Table 3. T2 Mature Plant Expression Organs/Tissues screened
Events Screened: n = 6 Events Expressing: n = 2

| X Root | L mature root |
|---|---|
| Table 4. | Promoter utility |
| Utility: | Among other uses this promoter sequence could be useful to |
| | improve: nitrogen and water loading and vasculature in limiting |
| | and non-limiting conditions, drought tolerance, biomass, protein |
| | content and composition. |

Notes: Peumans W J, Van Damme E J, Barre A, Rouge P. Classification of plant lectins in families
of structurally and evolutionary related proteins. Adv Exp Med Biol. 2001; 491: 27-54. Review.
PMID: 14533788
Barre A, Bourne Y, Van Damme E J, Peumans W J, Rouge P. Mannose-binding plant lectins:
different structural scaffolds for a common sugar-recognition process. Biochimie. 2001
July; 83(7): 645-51. Review. PMID: 11522393
Bouckaert J, Hamelryck T, Wyns L, Loris R. Novel structures of plant lectins and their complexes
with carbohydrates. Curr Opin Struct Biol. 1999 October; 9(5): 572-7. Review. PMID: 10508764
Van Damme E J, Zhang W, Peumans W J. Induction of cytoplasmic mannose-binding jacalin-
related lectins is a common phenomenon in cereals treated with jasmonate methyl ester. Commun
Agric Appl Biol Sci. 2004; 69(1): 23-31. PMID: 15560260

| | |
|---|---|
| Construct: | PT0769 |
| Promoter candidate I.D: | 15371914 |
| Events expressing: | 02, 03, 05, 06 |

Promoter Expression Report #293.PT0614

Promoter Tested In: Arabidopsis thaliana, Wassilewskija (WS) ecotype
Spatial expression summary:

| | |
|---|---|
| Primary Root | H cortex |

Observed expression pattern:
T1 Mature expression: No expression observed.
T2 Seedling expression: High GFP expression specific to root cortex cells.
T2 Mature expression: No expression detected.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

Table 1. T1 Mature Plant Expression Organs/Tissues screened
Events Screened: n = 6 Events Expressing: n = 0
X No GFP Expression Detected
Table 2. T2 Seedling Expression Tissues Screened
Events Screened: n = 6 Events Expressing: n = 6

| | |
|---|---|
| X Primary Root | □ epidermis □trichoblast □atrichoblast H cortex □ endodermis |
| | □ vascular □xylem □phloem □pericycle □quiescent |
| | □columella □ root cap □ root hairs |

Table 3. T2 Mature Plant Expression Organs/Tissues screened
Events Screened: n = 2 Events Expressing: n = 0
X No GFP Expression Detected

| | |
|---|---|
| Table 4. | Promoter utility |
| Utility: | Among other uses this promoter sequence could be useful to |
| | improve: water uptake and conductivity to vasculature and shoot, |
| | tolerance to drought and low soil water conditions, nitrogen uptake |
| | and utilization efficiency in limiting and non-limiting conditions. |
| Construct: | PT0614 |
| Promoter candidate I.D: | 13148301 |
| Events expressing: | 01-06 |

Promoter Expression Report #294.PT0621

Promoter Tested In: Arabidopsis thaliana, Wassilewskija (WS) ecotype
Spatial expression summary:

| | |
|---|---|
| Flower | H anther H tapetum H pollen |
| Leaf | L epidermis |
| Cotyledon | L mesophyll L vascular L epidermis |
| Primary Root | L epidermis |

Observed expression pattern:
T1 Mature expression: High GFP expression in developing pollen and tapetum cells of anthers.
Low expression in leaf epidermis.
T2 Seedling expression: Low GFP expression in root epidermis, and cotyledon vasculature,
mesophyll and epidermis.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

Table 1. T1 Mature Plant Expression Organs/Tissues screened
Events Screened: n = 6 Events Expressing: n = 5

| | |
|---|---|
| X Flower | □pedicel □receptacle □nectary □sepal □petal □filament H anther H |
| | tapetum H pollen □carpel □style □papillae □vascular □epidermis |
| | □stomata □trichome □silique |
| X Leaf | □petiole □mesophyll □vascular L epidermis □trichome □primordia |
| | □stomata □ stipule □margin |

Table 2. T2 Seedling Expression Tissues Screened
Events Screened: n = 6 Events Expressing: n = 5

| | |
|---|---|
| X Cotyledon | L mesophyll L vascular L epidermis □margin □petiole □ stomata |
| | □hydathode |
| X Primary Root | L epidermis □trichoblast □atrichoblast □ cortex □ endodermis |
| | □ vascular □xylem □phloem □pericycle □quiescent |
| | □columella □ root cap □ root hairs |

Table 3. T2 Mature Plant Expression Organs/Tissues screened
Events Screened: n = 2 Events Expressing: n = 2

| X Flower | □pedicel □receptacle □nectary □sepal □petal □filament H anther |
|---|---|
| | □pollen □carpel □style □papillae □vascular □epidermis □stomata |
| | □trichome □silique |
| X Leaf | □petiole □mesophyll □vascular H epidermis □trichome □primordia |
| | □stomata □stipule □margin |
| X Root | H mature root |
| Table 4. | Promoter utility |
| Utility: | Among other uses this promoter sequence could be useful to |
| | improve: male sterility, outbreeding, crossing and development of |
| | hybrids, CO₂ capture, sucrose loading and biomass, photosynthetic |
| | efficiency, growth rate, nitrogen assimilation. |

Notes: Evaluation and classification of RING-finger domains encoded by the Arabidopsis
genome.
Kosarev P, Mayer K F, Hardtke C S. Genome Biol. 2002; 3(4): research0016.1-research0016.12.
published online before print Mar. 14, 2002 PMCID: 115204 Abstract Full Text PDF-182K
Supplemental Data

| | |
|---|---|
| Construct: | PT0621 |
| Promoter candidate I.D: | 13148309 |
| Events expressing: | 01, 05, 06 |

Promoter Expression Report #295.PT0693

Promoter Tested In: Arabidopsis thaliana, Wassilewskija (WS) ecotype
Spatial expression summary:

| | |
|---|---|
| Flower | H anther |
| Leaf | L vascular L hydathode |
| Cotyledon | L vascular H epidermis H hydathode |
| Primary Root | H epidermis H root cap |

Observed expression pattern:
T1 Mature expression: High GFP expression specific to anthers in flowers. No pollen
expression. Low GFP expression in vascular tissues of leaf.
T2 Seedling expression: High GFP expression in epidermal cells at root transition zone
decreasing toward root tip.
T2 mature expression: GFP expressed in anthers and leaf vascular tissues.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

Inductions completed.

| | | | | | |
|---|---|---|---|---|---|
| Treatment: | Age: | Gen: | Time Points: | Events | Response |
| | | | | Screened/ | |
| | | | | Response | |
| 1. Drought | 4 wks | T2 | ≈1.0% moisture | 6/0 | No |

Table 1. T1 Mature Plant Expression Organs/Tissues screened
Events Screened: n = 6 Events Expressing: n = 6

| | |
|---|---|
| X Flower | □pedicel □receptacle □nectary □sepal □petal □filament H anther |
| | □tapetum □pollen □carpel □style □papillae □vascular □epidermis |
| | □stomata □trichome □silique |
| X Leaf | □petiole □mesophyll L vascular □epidermis □trichome □primordia |
| | □stomata □stipule □margin L hydathode |

Table 2. T2 Seedling Expression Tissues Screened
Events Screened: n = 6 Events Expressing: n = 5

| | |
|---|---|
| X Cotyledon | □mesophyll L vascular H epidermis □margin □petiole □ |
| | stomata H hydathode |
| X Primary Root | H epidermis □trichoblast □atrichoblast □ cortex □ |
| | endodermis □ vascular □xylem □phloem □pericycle □quiescent |
| | □columella H root cap □ root hairs |

Table 3. T2 Mature Plant Expression Organs/Tissues screened
Events Screened: n = 2 Events Expressing: n = 2

| | |
|---|---|
| X Flower | □pedicel □receptacle □nectary □sepal □petal □filament H anther |
| | □pollen □carpel □style □papillae □vascular □epidermis □stomata |
| | □trichome □silique |
| X Leaf | □petiole □mesophyll L vascular □epidermis □trichome □primordia |
| | □stomata □stipule □margin |

Table 3. Induction Screens
No drought induction.

| | |
|---|---|
| Table 4. | Promoter utility |
| Utility: | Among other uses this promoter sequence could be useful to |
| | improve: crossing and outbreeding, male sterility, seedling |
| | establishment and growth, tolerance to low soil water content and |
| | drought, nitrogen use efficiency and uptake of nitrogen in limiting |
| | and non-limiting conditions. |
| Construct: | PT0693 |
| Promoter candidate I.D: | 15371530 |
| Events expressing: | 03, 04, 05, 06, 08 |

Promoter Expression Report#303.PT0761

Promoter Tested In: Arabidopsis thaliana, Wassilewskija (WS) ecotype
Spatial expression summary:

| | |
|---|---|
| Primary Root | H epidermis H root cap |

Observed expression pattern:
T1 Mature expression: No expression observed.
T2 Seedling expression: High GFP expression throughout seedling root epidermis.
T2 Mature expression: No expression observed.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature X T2 Seedling XT2 Mature □T3 Seedling |

Table 1. T1 Mature Plant Expression Organs/Tissues screened
Events Screened: n = 3 Events Expressing: n = 0
No GFP Expression Detected
Table 2. T2 Seedling Expression Tissues Screened
Events Screened: n = 6 Events Expressing: n = 3

| | |
|---|---|
| X Primary Root | H epidermis □trichoblast □atrichoblast □ cortex □ endodermis |
| | □ vascular □xylem □phloem □pericycle □quiescent |
| | □columella H root cap □ root hairs |

Table 3. T2 Mature Plant Expression Organs/Tissues screened
Events Screened: n = 2 Events Expressing: n = 0
X No GFP Expression Detected

| | |
|---|---|
| Table 4. | Promoter utility |
| Utility: | Among other uses this promoter sequence could be useful to |
| | improve: nitrogen and mineral ion uptake and loading to cortex and |
| | vasculature, water uptake and loading to cortex and xylem, |
| | tolerance of plants to low nitrogen and drought conditions, and |
| | protection against root nematodes and pathogens. |
| Construct: | PT0761 |
| Promoter candidate I.D: | 15371875 |
| Events expressing: | −01, −03, −06 |

Promoter Expression Report #322.PT1016

Promoter Tested In: Arabidopsis thaliana, Wassilewskija (WS) ecotype
Spatial expression summary:

| | |
|---|---|
| Flower | H receptacle H sepal H vascular |
| Hypocotyl | L vascular |
| Cotyledon | L vascular L hydathode |
| Primary Root | H epidermis |
| Root | H mature root |

Observed expression pattern:
T1 Mature expression: High GFP expression in vasculature of flowers at abscission zone.
T2 Seedling expression: High GFP expression in epidermis. Low GFP expression in vasculature
of hypocotyl and cotyledons.
T2 Mature expression: High GFP expression in roots and in vasculature of flowers at abscission
zone.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 6 Events Expressing: n = 2

| | |
|---|---|
| X Flower | □pedicel H receptacle □nectary H sepal □petal □filament |
| | □anther □tapetum □pollen □carpel □style □papillae H |
| | vascular □epidermis □stomata □trichome □silique |

Table 2. T2 Seedling Expression Tissues Screened
Events Screened: n = 5 Events Expressing: n = 6

| | |
|---|---|
| X Hypocotyl | □epidermis □cortex L vascular □xylem □phloem □stomata |
| X Cotyledon | □mesophyll L vascular □ epidermis □margin □petiole □ stomata |
| | L hydathode |
| X Primary Root | H epidermis □trichoblast □atrichoblast □ cortex □ endodermis |
| | □ vascular □xylem □phloem □pericycle □quiescent □columella |
| | □ root cap □ root hairs |

Table 3. T2 Mature Plant Expression Organs/Tissues screened
Events Screened: n = 6 Events Expressing: n = 5

| X Flower | □pedicel H receptacle □nectary □sepal □petal □filament |
|---|---|
| | □anther □pollen □carpel □style □papillae H |
| | vascular □epidermis □stomata □trichome □silique |
| X Root | H Mature root |
| Table 4. | Promoter utility |
| Utility: | Among other uses this promoter sequence could be useful to |
| | improve: development and structure of flowers, carpel and seed |
| | number, seed yield, breeding biology (outcrossing), water and |
| | nitrogen uptake from soil, water and nitrogen transport and use |
| | efficiency, flower, fruit and seed abscission, and post-harvest |
| | maturation of fruit and seed. |
| Construct: | PT1016 |
| Promoter candidate I.D: | 22204494 |
| Events expressing: | 02-06 |

Promoter Expression Report #211.PT0695

Promoter Tested In: Arabidopsis thaliana, Wassilewskija (WS) ecotype
Spatial expression summary:

| | |
|---|---|
| Embryo | L late L mature |
| Hypocotyl | L epidermis |
| Cotyledon | H epidermis H petiole |

Observed expression pattern:
T1 mature: Embryo specific GFP expression. GFP expressed in developing and mature embryo.
T2 seedling: GFP expressed in epidermis of cotyledon.
T2 mature: Embryo expression confirmed. No GFP expression detected in other organs.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 mature□T3 Seedling |

Inductions completed.

| | | | | | |
|---|---|---|---|---|---|
| Treatment | Age: | Gen: | Time points: | Events | Response: |
| | | | | Screened/ | |
| | | | | Response | |
| 1. Drought | 4 wks | T2 | 8 days ≈ | 3/0 | No |
| | | | 1.0% moisture | | |

Inducible expression summary:

| | | | |
|---|---|---|---|
| Treatment: | Time point induced: | Organs induced: | Tissues induced: |

Table 1. T1 Mature Plant Expression Organs/Tissues screened
Events Screened: n = 2 Events Expressing: n = 2

| | |
|---|---|
| X Embryo | □suspensor □preglobular □globular □heart □torpedo |
| | L late L mature □provascular □hypophysis □radicle |
| | □cotyledons □hypocotyl |

Table 2. T2 Seedling Expression Tissues Screened
Events Screened: n = 3 Events Expressing: n = 1
Seedlings expressing/Seedlings screened
Event-01: 0/6
Event-02: 0/6
Event-03: 3/6
GFP Expression Detected

| | |
|---|---|
| X Hypocotyl | L epidermis □cortex □vascular □xylem □phloem |
| X Cotyledon | □stomata □mesophyll □vascular H epidermis □margin |
| | H petiole □stomata □hydathode |

Table 3. T2 Mature Plant Expression Organs/Tissues screened
Events Screened: n = 1 Events Expressing: n = 1
X No GFP Expression Detected
T2 mature scan. Embryo expression confirmed. No germination in second event.

| | |
|---|---|
| Table 4. | Promoter utility |
| Utility: | Among other uses this promoter sequence could be useful to |
| | improve: size, seed size, seed weight, seed yield, seed |
| | composition, germination timing, germination efficiency, |
| | germination rate, root growth, leaf angle, light capture and |
| | source strength in shade and low light. |
| Construct: | PT0695 |
| Promoter candidate I.D: | 15371536 |
| Lines expressing: | 01, 03 |

Promoter Expression Report #225.PT0879

Promoter Tested In: Arabidopsis thaliana, Wassilewskija (WS) ecotype
Spatial expression summary:

| | |
|---|---|
| Embryo | H late H mature H suspensor H provascular |
| | H hypophysis H radicle H cotyledons |

Observed expression pattern:
T1 mature: Embryo specific GFP expression. High GFP expression throughout mature embryo.
GFP preferentially expressed at root cap.
T2 seedling: No expression observed.
T2 mature: Embryo specific expression confirmed.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling XT2 Mature □ T3 Seedling |

Inductions completed.

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | 8 days no water | 2/0 | No |
| 2. Drought | 4 wks | T2 | ~1.0% moisture | 6/0 | No |

Inducible expression summary:

| | | | |
|---|---|---|---|
| Treatment: | Time point induced: | Organs induced: | Tissues induced: |

Table 1. T1 Mature Plant Expression Organs/Tissues screened
Events Screened: n = 6 Events Expressing: n = 3

| | |
|---|---|
| X Embryo | H suspensor □preglobular □globular □heart □torpedo |
| | H late H mature H provascular H hypophysis H radicle |
| | H cotyledons |

Table 2. T2 Seedling Expression Tissues Screened
Events Screened: n = 2 Events Expressing: n = 0
No GFP Expression Detected
Table 3. T2 Mature Plant Expression Organs/Tissues screened
Events Screened: n = 2 Events Expressing: n = 2

| X Embryo | □suspensor □preglobular □globular □heart □torpedo |
|---|---|
| | H late H mature □provascular □hypophysis □radicle |
| | □cotyledons □root meristem □shoot meristem |
| Table 3. | Promoter utility |
| Utility: | Among other uses this promoter sequence could be useful to |
| | improve: embryo size, seed size, seed yield, seed composition, |
| | more efficient germination, faster germination, faster seedling |
| | emergence and establishment, faster seedling growth, and more |
| | robust seedlings. |
| Construct: | PT0879 |
| Promoter candidate I.D: | 15371602 |
| Lines expressing: | 02, 05, 06 |

Promoter Expression Report #246.PT0738

Promoter Tested In: Arabidopsis thaliana, Wassilewskija (WS) ecotype
Spatial expression summary:

| | |
|---|---|
| Flower | H pedicel H receptacle |
| Primary Root | H epidermis H cortex H endodermis H vascular |
| | H pericycle |

Observed expression pattern:
T1 Mature: None observed.
T2 Seedling: High GFP expression throughout seedling root. Highest GFP expression at
transition zone decreasing toward root tip.
T2 Mature: High GFP expression in pedicels of flowers at the inflorescences.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Inductions completed.

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | ≈1.0% moisture | 6/0 | No |

Table 1. T1 Mature Plant Expression Organs/Tissues screened
Events Screened: n = 3 Events Expressing: n = 0
No GFP Expression Detected
Table 2. T2 Seedling Expression Tissues Screened
Events Screened: n = 3 Events Expressing: n = 3
Seedlings expressing/Seedlings screened
Event-01: 4/6
Event-02: 3/6
Event-02: 5/6

| | |
|---|---|
| X Primary Root | H epidermis □trichoblast □atrichoblast H cortex H endodermis |
| | H vascular □xylem □phloem H pericycle □quiescent |
| | □columella □ root cap □ root hairs |

Table 3. T2 Mature Plant Expression Organs/Tissues screened
Events Screened: n = 2 Events Expressing: n = 2

| X Flower | H pedicel H receptacle □nectary □sepal □petal |
|---|---|
| | □filament □anther □pollen □carpel □style |
| | □papillae □vascular □epidermis □stomata |
| | □trichome □silique |
| Table 4. | Promoter utility |
| Utility: | Among other uses this promoter sequence could be useful to |
| | improve: nitrogen use efficiency and nitrogen loading for transport |
| | in low and non-limiting nitrogen conditions, enhanced water uptake |
| | in drought and non-limiting water environments, and water loading |
| | to vasculature and transport to shoot, floral morphology, flower |
| | structure, pollination and breeding biology, senescence, flowers and |
| | fruit abscission and flower and fruit drop. |
| Construct: | PT0738 |
| Promoter candidate I.D: | 15371758 |
| Events expressing: | 01, 02, 03 |

Promoter Expression Report #247.PT0834

Promoter Tested In: Arabidopsis thaliana, Wassilewskija (WS) ecotype
Spatial expression summary:

| | |
|---|---|
| Flower | H anther |
| Leaf | L mesophyll L epidermis |
| Root | H mature root |
| Primary Root | H epidermis H root hairs |

Observed expression pattern:
T1 Mature: High GFP expression in anther walls and roots. Low GFP expression in leaf
epidermis and mesophyll.
T2 Seedling: High GFP expression in epidermis of roots.
T2 Mature: GFP expression detected in anthers. Low root GFP expression.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

Inductions completed.

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | ≈1.0% moisture | 6/0 | No |

Inducible expression summary:

| | | | |
|---|---|---|---|
| Treatment: | Time point induced: | Organs induced: | Tissues induced: |

Table 1. T1 Mature Plant Expression Organs/Tissues screened
Events Screened: n = 3 Events Expressing: n = 2

| | |
|---|---|
| X Flower | □pedicel □receptacle □nectary □sepal □petal □filament |
| | H anther □tapetum □pollen □carpel □style □papillae |
| | □vascular □epidermis □stomata □trichome □silique |
| X Leaf | □petiole L mesophyll □vascular L epidermis □trichome |
| | □primordia □stomata □stipule □margin |
| X Root | H mature root |

Table 2. T2 Seedling Expression Tissues Screened
Events Screened: n = 2 Events Expressing: n = 2
Seedlings expressing/Seedlings screened
Event 01: 3/6
Event 02: 4/6

| | |
|---|---|
| X Primary Root | H epidermis □trichoblast □atrichoblast □ cortex □ endodermis |
| | □ vascular □xylem □phloem □pericycle □quiescent |
| | □columella □ root cap H root hairs |

Table 3. T2 Mature Plant Expression Organs/Tissues screened
Events Screened: n = 2 Events Expressing: n = 2

| X Flower | □pedicel □receptacle □nectary □sepal □petal □filament H anther |
|---|---|
| | □pollen □carpel □style □papillae □vascular □epidermis □stomata |
| | □trichome |
| | □silique |
| X Root | L mature root |
| Table 3. | Promoter utility |
| Utility: | Among other uses this promoter sequence could be useful to |
| | improve: male sterility, outbreeding, crossing, C0₂ capture, sucrose |
| | loading of seeds, leaf area and photosynthetic capacity, growth rate |
| | and biomass. |
| Construct: | PT0834 |
| Promoter candidate I.D: | 15371521 |
| Events expressing: | 01, 02 |

Promoter Expression Report #254.PT0746

Promoter Tested In: Arabidopsis thaliana, Wassilewskija (WS) ecotype
Spatial expression summary:

| | |
|---|---|
| Primary Root | L epidermis L cortex H pericycle |
| Mature root | L epidermis L cortex L vascular H pericycle |

Observed expression pattern:
T1 Mature expression: High GFP expression in mature root. GFP expression observed in all
cells of lower root near root tip. GFP specific to pericycle cells in upper roots.
T2 Seedling expression: High GFP expression in pericycle cells at transition zone increasing
expression in epidermis and cortex toward root tip.
T2 Mature: High GFP expression in roots.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 6 Events Expressing: n = ≧1

| | |
|---|---|
| X Root | L epidermis □trichoblast □atrichoblast L cortex □ endodermis |
| | L vascular □xylem □phloem H pericycle □quiescent |
| | □columella □ root cap □ root hairs |

Table 2. T2 Seedling Expression Tissues Screened
Events Screened: n = 6 Events Expressing: n = 2
Seedlings expressing/Seedlings screened
Event-01: 3/6
Event-02: 4/6

| | |
|---|---|
| X Primary Root | L epidermis □trichoblast □atrichoblast L cortex □ endodermis |
| | □ vascular □xylem □phloem H pericycle □quiescent |
| | □columella □ root cap □ root hairs |

Table 3. T2 Mature Plant Expression Organs/Tissues screened
Events Screened: n = 2 Events Expressing: n = 2

| X Root | H mature root |
|---|---|
| Table 4. | Promoter utility |
| Utility: | Among other uses this promoter sequence could be useful to |
| | improve: uptake and loading to the vasculature in shoot, water |
| | capture from soil, and nitrogen uptake and loading, all in limiting |
| | and non-limiting environments. |
| Construct: | PT0746 |
| Promoter candidate I.D: | 15371812 |
| Events expressing: | 01, 05 |

Promoter Expression Report #299.PT0607

Promoter Tested In: Arabidopsis thaliana, Wassilewskija (WS) ecotype
Spatial expression summary:

| | |
|---|---|
| Ovule | Post-fertilization: H outer integument H endothelium H seed coat |
| Root | H mature root |

Observed expression pattern:
T1 Mature expression: High GFP expression in seed coat of developing seed. Seed coat specific
GFP expression.
T2 Seedling expression: None observed.
T2 Mature expression: GFP expressed in seed coat and roots.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling X T2 Mature □T3 Seedling |

Table 1. T1 Mature Plant Expression Organs/Tissues screened
Events Screened: n = 6 Events Expressing: n = 3

| | |
|---|---|
| X Ovule | Pre-fertilization: □primordia □inner integument □outer integument |
| | □embryo sac □funiculus □chalaza □micropyle □gametophyte |
| | Post-fertilization: □zygote □suspensor □ embryo sack □funiculus |
| | □inner integument H outer integument H endothelium H seed coat |
| | □primordia □chalaza □micropyle □early endosperm □mature |
| | endosperm □embryo |

Table 2. T2 Seedling Expression Tissues Screened
Events Screened: n = 6 Events Expressing: n = 0
No GFP Expression Detected
Table 3. T2 Mature Plant Expression Organs/Tissues screened
Events Screened: n = 2 Events Expressing: n = 2

| X Root | H mature root |
|---|---|
| Table 4. | Promoter utility |
| Utility: | Among other uses this promoter sequence could be useful to |
| | improve: seed size, seed weight, seed shape, seed germination |
| | timing, germination efficiency, resistance to seed fungal and |
| | bacterial pathogens, nitrogen and water utilization and tolerance to |
| | low nitrogen and drought environments. |
| Construct: | PT0607 |
| Promoter candidate I.D: | 13148287 |
| Events expressing: | 02, 05, 06 |

Promoter Expression Report#300.PT0861

Promoter Tested In: Arabidopsis thaliana, Wassilewskija (WS) ecotype
Spatial expression summary:

| | |
|---|---|
| Flower | H vascular H pedicel H receptacle |
| Hypocotyl | H vascular |
| Cotyledon | H vascular |
| Rosette Leaf | H vascular |
| Primary Root | H vascular H xylem H phloem H pericycle H endodermis |
| Root | H mature root |

Observed expression pattern:
T1 Mature expression: High GFP expression specific to vascular tissues at inflorescences and
root.
T2 Seedling expression: High GFP expression in vascular cells throughout organs of seedling.
T2 Mature expression: High GFP expression specific to vascular tissues at inflorescences and
root.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

Table 1. T1 Mature Plant Expression Organs/Tissues screened
Events Screened: n = 3 Events Expressing: n = 2

| | |
|---|---|
| X Flower | H pedicel H receptacle □nectary □sepal □petal □filament □anther |
| | □tapetum □pollen □carpel □style □papillae H vascular □epidermis |
| | □stomata □trichome |
| | □silique |

Table 2. T2 Seedling Expression Tissues Screened
Events Screened: n = 6 Events Expressing: n = 2

| | |
|---|---|
| X Hypocotyl | □epidermis □cortex H vascular □xylem □phloem □stomata |
| X Cotyledon | □mesophyll H vascular □ epidermis □margin □petiole □ |
| | stomata |
| | □hydathode |
| X Rosette Leaf | □mesophyll H vascular □epidermis □trichome □petiole |
| | □primordia □stomata □stipule □margin □hydathode |
| X Primary Root | □ epidermis □trichoblast □atrichoblast □ cortex H |
| | endodermis |
| | H vascular H xylem H phloem H pericycle □quiescent |
| | □columella □ root cap □ root hairs |

Table 3. T2 Mature Plant Expression Organs/Tissues screened
Events Screened: n = 6 Events Expressing: n = 5

| X Flower | □pedicel □receptacle □nectary □sepal □petal □filament □anther |
|---|---|
| | □pollen □carpel □style □papillae H vascular □epidermis □stomata |
| | □trichome |
| | □silique |
| X Root | H mature root |
| Table 4. | Promoter utility |
| Utility: | Among other uses this promoter sequence could be useful to |
| | improve: water loading from root to shoot, water transport to shoot |
| | and flower, tolerance to drought, loss of flowers, fruits and seeds to |
| | wilting, drooping and abscission, nitrogen translocation from root to |
| | shoot, nitrogen uptake and loading, and fruit and seed yield under |
| | limiting and non-limiting soil water and nitrogen environments. |
| Construct: | PT0861 |
| Promoter candidate I.D: | 15371947 |
| Events expressing: | 01-06 |

Promoter Expression Report#318.PT0760

Promoter Tested In: Arabidopsis thaliana, Wassilewskija (WS) ecotype
Spatial expression summary:

| | |
|---|---|
| Primary Root | H epidermis |

Observed expression pattern:
T1 Mature expression: None observed.
T2 Seedling expression: Seedling specific root expression. High GFP expression specific to
epidermal cells of root.
T2 Mature expression: None detected.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 3 Events Expressing: n = 0
No GFP Expression Detected
Table 2. T2 Seedling Expression Tissues Screened
Events Screened: n = 3 Events Expressing: n = 2
Seedlings expressing/Seedlings screened
Event-01: 5/6
Event-02: 4/6

| | |
|---|---|
| X Primary Root | H epidermis □trichoblast □atrichoblast □ cortex □ endodermis |
| | □ vascular □xylem □phloem □pericycle □quiescent |
| | □columella □ root cap □ root hairs |

Table 3. T2 Mature Plant Expression Organs/Tissues screened
Events Screened: n = 6 Events Expressing: n = 0
No GFP Expression Detected

| | |
|---|---|
| Table 4. | Promoter utility |
| Utility: | Among other uses this promoter sequence could be useful to |
| | improve: Water uptake, nitrogen uptake, loading of water and |
| | nitrogen to cortex and vasculature, tolerance to drought and low |
| | levels of soil nitrogen, and protection against soil nematodes and |
| | fungal and bacterial pathogens. |
| Construct: | PT0760 |
| Promoter candidate I.D: | 15371872 |
| Events expressing: | 01, 03 |

Promoter Expression Report#319.PT0878

Promoter Tested In: Arabidopsis thaliana, Wassilewskija (WS) ecotype
Spatial expression summary:

| | |
|---|---|
| Primary Root | H epidermis H cortex H endodermis |
| Root | H mature root |

Observed expression pattern:
T1 Mature expression: None observed.
T2 Seedling expression: High GFP expression in ground cells - epidermis, cortex and endodermis
of seedling root. Root specific GFP expression. Not in vascular bundle.
T2 mature: High GFP expression in roots.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Table 1. T1 Mature Plant Expression Organs/Tissues screened
Events Screened: n = 3 Events Expressing: n = 0
No GFP Expression Detected
Table 2. T2 Seedling Expression Tissues Screened
Events Screened: n = 6 Events Expressing: n = 2

| | |
|---|---|
| X Primary Root | H epidermis □trichoblast □atrichoblast H cortex H endodermis □ |
| | vascular □xylem □phloem □pericycle □quiescent □columella □ root |
| | cap □ root hairs |

Table 3. T2 Mature Plant Expression Organs/Tissues screened
Events Screened: n = 6 Events Expressing: n = 5

| X Root | H mature root |
|---|---|
| Table 4. | Promoter utility |
| Utility: | Among other uses this promoter sequence could be useful to |
| | improve: nitrogen use efficiency in lower/non-limiting nitrogen |
| | environments, enhanced water uptake in drought and non-limiting |
| | water environments, and protection against soil-borne nematodes, |
| | root worms, fungal and bacterial pathogens. |
| Construct: | PT0878 |
| Promoter candidate I.D: | 15371944 |
| Events expressing: | 01, 02, 03, 04, 06 |

Promoter Expression Report #519.YP2532

Promoter Tested In: Arabidopsis thaliana, Wassilewskija (WS) ecotype
Spatial expression summary:

| | |
|---|---|
| Flower | H stomata H nectary H petal |
| Silique | H stomata |
| Stem | H stomata |
| Leaf | H stomata |
| Primary Root | H cortex H epidermis H root hairs L root cap |

Observed expression pattern:
T1 Mature expression: GFP expressed in guard cells throughout aerial organs. High GFP
expression in flowers stems and leaves. In flowers, GFP also highly expressed in nectary and

petals.

T2 Seedling expression: High GFP expression in root epidermis cells including root hairs.
T2 Mature expression: GFP expressed in root epidermis.

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | X T1 Mature X T2 Seedling X T2 Mature □T3 Seedling |

T1 Mature Plant Expression Organs/Tissues screened
Events Screened: n = 6 Events Expressing: n = 3 (01, 04, 05)

| | |
|---|---|
| X Flower | □pedicel □receptacle H nectary □sepal H petal □filament □anther |
| | □tapetum □pollen □carpel □style □ stigma □vascular □epidermis H |
| | stomata □trichome |
| | □silique |
| X Silique | □stigma □style □carpel □septum □placentae □funiculus □transmitting |
| | tissue □vascular □epidermis H stomata □ abscission zone □ovule |
| X Stem | □epidermis □cortex □interfascicular region □vascular □xylem □phloem |
| | □pith H stomata □trichome |
| X Leaf | □petiole □mesophyll □vascular □epidermis □trichome □primordia H |
| | stomata □stipule □margin |

T2 Seedling Expression Tissues Screened
Events Screened: n = 5 Events Expressing: n = 3 (02, 04, 05)

| | |
|---|---|
| X Primary Root | H epidermis □trichoblast □atrichoblast H cortex □ endodermis |
| | □ vascular □xylem □phloem □pericycle □quiescent center □ root |
| | meristem □columella L root cap H root hairs |

T2 Mature Plant Expression Organs/Tissues screened
Events Screened: n = 5 Events Expressing: n = 3 (01, 02, 05)

| X Root | H mature root |
|---|---|
| Promoter utility | |
| Utility: | Among other uses this promoter sequence could be useful to |
| | improve: the uptake of water and nutrients and to provide a more |
| | rapid establishment of seedlings by increasing nutrient uptake. |
| Construct: | YP2532 |
| Promoter candidate I.D: | 40983432 |
| Events expressing: | 01, 02, 05 |

Promoter Expression Report #696.YP2573

Promoter Tested In: Arabidopsis thaliana, Wassilewskija (WS) ecotype
Spatial expression summary:
Drought-induced expression in roots and flowers.
ABA-induced expression in seedlings.
Observed expression pattern:
T1 mature: No expression observed
T2 seedling: No expression observed
T2 Mature: No expression observed

| | |
|---|---|
| Source Promoter Organism: | Arabidopsis thaliana, Columbia (Col) ecotype |
| Vector: | pNewbin4-HAP1-GFP |
| Marker Type: | GFP-ER |
| Generation Screened: | XT1 Mature XT2 Seedling XT2 Mature □T3 Seedling |

Inductions completed:

| | | | | | |
|---|---|---|---|---|---|
| Treatment: | Age: | Gen: | Time points: | Events | Response: |
| | | | | Screened/ | |
| | | | | Response | |
| 1. Drought | 4 wks | T2 | 2% Soil | 4/2 | Moderate |
| | | | Moisture | | |
| 2. ABA [100 μM] | 10 days | T2 | 24 hours | 5/4 | Moderate |

Inducible expression summary:

| | | | |
|---|---|---|---|
| Treatment: | Time point induced: | Organs induced: | Tissues induced: |
| 1. Drought | 2% Soil | Root | |
| | Moisture | Flowers | |
| 2. ABA [100 μM] | 24 hr | Seedlings | |

T1 Mature Plant Expression Organs/Tissues screened
Events Screened: n = 5 Events Expressing: n = 0
No GFP Expression Detected

| | |
|---|---|
| □ Flower | □pedicel □receptacle □nectary □sepal □petal □filament □anther |
| | □pollen □carpel □style □papillae □vascular □epidermis □ stomata |
| | □trichome |
| | □silique |
| □ Silique | □stigma □style □carpel □septum □placentae □transmitting tissue |
| | □vascular □epidermis □ stomata □abscission zone □ovule |
| □ Ovule | Pre-fertilization: □primordia □inner integument □outer integument |
| | □embryo sac □funiculus □chalaza □micropyle □gametophyte |
| | Post-fertilization: □zygote □suspensor □ embryo sack □inner |
| | integument □outer integument □endothelium □seed coat □primordia |
| | □chalaza □micropyle □early endosperm □mature endosperm □embryo |
| □ Embryo | □suspensor □preglobular □globular □heart □torpedo □late □mature |
| | □provascular □hypophysis □radicle □cotyledons □hypocotyl |
| □ Stem | □epidermis □cortex □vascular □xylem □phloem □pith □ stomata |
| | □trichome |
| □ Leaf | □petiole □mesophyll □vascular □epidermis □trichome □primordia |
| | □ stomata □stipule □margin |
| □ Shoot apical | □ Shoot apical meristem □ Flower primordium |
| meristem | |

T2 Seedling Expression Tissues Screened
Events Screened: n = 5 Events Expressing: n = 0
No GFP Expression Detected

| | |
|---|---|
| □ Hypocotyl | □epidermis □cortex □ vascular □xylem □phloem □stomata |
| □ Cotyledon | □mesophyll □vascular □ epidermis □margin □stomata |
| | □hydathode |
| □ Rosette Leaf | □mesophyll □vascular □epidermis □trichome □petiole |
| | □primordia □stomata □stipule □margin □hydathode |
| □ Primary Root | □ epidermis □trichoblast □atrichoblast □cortex □endodermis |
| | □ vascular □xylem □phloem □ pericycle □quiescent |
| | □columella □root cap □root hairs |
| □ Lateral root | □epidermis □trichoblast □atrichoblast □cortex □endodermis |
| | □ initials □flanking cells □vascular □lateral root cap |
| □ Shoot apical meristem | □Shoot apical meristem |

T2 Mature Plant Expression Organs/Tissues screened
Events Screened: n = 5 Events Expressing: n = 0
No GFP Expression Detected
Inductions Table Organs/Tissues screened
100 μM ABA Treatment
Events Screened: n = 5 Events Expressing: n = 4 (01, 02, 04, 05)
Drought Induction 2% Soil Moisture Whole Plant
Events Screened: n = 4 Events Expressing: n = 2 (01, 05)

| | |
|---|---|
| Promoter utility | |
| Utility: | Among other things, this promoter could be engineered to |
| | promote drought-tolerance in plants by increasing water |
| | uptake or reducing water loss during drought-stress. |
| Construct: | YP2573 |
| Promoter candidate I.D: | 29223801 |
| cDNA I.D: | 36536957 |
| Lines expressing: | 01, 02, 04, 05 |

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

1. An isolated nucleic acid molecule that shows at least 80% sequence identity to any one of SEQ ID Nos. 1-22, wherein said nucleic acid molecule comprises a regulatory region that directs transcription of an operably linked heterologous polynucleotide.

2. The nucleic acid of claim 1, wherein said regulatory region shows at least 85 percent sequence identity to any one of SEQ ID NOs: 1-22.

3. The nucleic acid of claim 1, wherein said regulatory region has at least 90 percent sequence identity to any one of SEQ ID NOs: 1-22.

4. The isolated nucleic acid molecule of claim 1, wherein said regulatory region comprises at least one member selected from the group consisting of a promoter, an enhancer and an intron.

5. A vector construct comprising:
a) a first nucleic acid molecule according to claim 1; and
b) a second nucleic acid molecule to be transcribed,
wherein said first and second nucleic acid molecules are heterologous to each other and are operably linked.

6. The vector construct according to claim 5, wherein said first nucleic acid consists of the nucleic acid molecule set forth in any one of SEQ ID NOs: 1-22.

7. A vector according to claim 5, wherein said second nucleic acid molecule comprises a nucleic acid sequence that encodes a polypeptide.

8. A vector according to claim 7, wherein said second nucleic acid molecule is operably linked to said first nucleic acid molecule in the sense orientation.

9. A vector according to claim 8, wherein said second nucleic acid molecule is transcribed into an RNA molecule that expresses the polypeptide encoded by said second nucleic acid molecule.

10. A vector according to claim 7, wherein said second nucleic acid molecule is operably linked to said first nucleic acid molecule in the antisense orientation.

11. A vector according to claim 10, wherein said second nucleic acid molecule is transcribed into an antisense RNA molecule.

12. A vector according to claim 5, wherein said second nucleic acid molecule is transcribed into an interfering RNA against an endogenous gene.

13. A plant or plant cell transformed with:
a) a nucleic acid molecule according to claim 1 that is operably linked to a heterologous polynucleotide, or
b) a vector construct according to claim 5.

14. The plant or plant cell of claim 13, wherein said first nucleic acid molecule consists of the sequence set forth in any one of SEQ ID NOs: 1-22.

15. A plant or plant cell transformed with a vector construct according to any one of claim 7, 8, 9, 10 or 11.

16. A method of directing transcription by combining, in an environment suitable for transcription:
a) a first nucleic acid molecule according to claim 1; and
b) a second nucleic acid molecule to be transcribed;
wherein said first and second nucleic acid molecules are heterologous to each other and operably linked.

17. The method of claim 16, wherein said first nucleic acid molecule consists of a sequence according to any one of SEQ ID NOs: 1-22.

18. The method of claim 16 or 17, wherein said operably linked first and second nucleic acid molecules are inserted into a plant cell and said plant cell is regenerated into a plant.

19. A plant comprising a vector according to claim 5.

20. A plant according to claim 19, wherein said second nucleic acid molecule codes for a polypeptide of agronomic interest.

21. A transgenic plant according to claim 20, wherein said nucleic acid molecule and said transcribable nucleic acid molecule are heterologous to each other.

22. A seed of a plant according to claim 20 or 21.

23. A method of expressing an exogenous coding region in a plant comprising:
(a) transforming a plant cell with a vector of claim 8;
(b) regenerating a stably transformed plant from the transformed plant cell of step (a); and
(c) selecting plants containing a transformed plant cell,
wherein expression of the vector gene results in production of a polypeptide encoded by said second nucleic acid.

24. A method of altering the expression of a gene in a plant comprising:
a) transforming a plant cell with a nucleic acid molecule according to claim 1 that is operably linked to a heterologous polynucleotide, and
b) regenerating stably transformed plants from said transformed plant cell.

25. A plant prepared according to the method of claim 23 or 24.

26. Seed from a plant according to claim 25.

27. A method of producing a transgenic plant, said method comprising:
(a) introducing into a plant cell:
(i) an isolated polynucleotide comprising a nucleic acid according to claim 1 that is operably linked to a heterologous polynucleotide, or
(ii) a vector according to claim 5, and
(b) growing a plant from said plant cell.

28. The method of claim 27, wherein said heterologous polynucleotide comprises a nucleic acid sequence encoding a polypeptide.

29. The method of claim 27, wherein said heterologous polynucleotide is operably linked to said regulatory region in the antisense orientation.

30. The method of claim 27, wherein said heterologous polynucleotide is transcribed into an interfering RNA.
